(19) United States
(12) Patent Application Publication
(10) Pub. No.: US 2024/0139206 A1
(43) Pub. Date: May 2, 2024

(54) METHODS OF TREATMENT

(71) Applicant: Kinoxis Therapeutics Pty Ltd, Camberwell (AU)

(72) Inventors: Michael Thomas BOWEN, Camberwell (AU); Iain Stewart MCGREGOR, Camberwell (AU)

(21) Appl. No.: 18/547,377

(22) PCT Filed: Mar. 18, 2022
(86) PCT No.: PCT/AU2022/050244

§ 371 Date: Aug. 22, 2023

(30) Foreign Application Priority Data
Mar. 18, 2021 (AU) 2021900789

(51) Int. Cl. A61K 31/5517; A61P 25/20

(52) U.S. Cl. CPC A61K 31/5517; A61P 25/20

(57) ABSTRACT

The present invention provides methods for treating or preventing aggression, agitation, irritability and/or anger in a subject comprising administrating to a subject in need thereof a therapeutically effective amount of a compound of Formula (I).

### FIELD OF THE INVENTION

The present invention relates to methods for the treatment and prevention of behavioural disorders and compositions for use in the same.

### RELATED APPLICATION

This application claims priority from Australian provisional application AU 2021900789, the entire contents of which are hereby incorporated by reference.

### BACKGROUND OF THE INVENTION

Aggression can develop as a symptom of a range of disorders, diseases, and conditions, including neurodegenerative diseases, neurodevelopmental disorders, stroke and traumatic brain injury. Prevalence of aggression in many of these disorders is very high. For instance, aggression is present in as many as 35% of patients following stroke, 29% of patients following a traumatic brain injury, and 40% of patients with Alzheimer's disease.

Despite the pervasiveness and debilitating nature of aggression in these conditions, attempts to develop a serenic, a drug which specifically targets aggression, agitation and irritability, have been unsuccessful thus far. As a result, drugs with only minimal efficacy and considerable side effects are often prescribed for managing agitation, irritability and aggression within the context of certain conditions. For example, the antipsychotics risperidone and aripiprazole, with their myriad adverse effects, are prescribed to manage irritability in children with autism spectrum disorder and are used, off-label, at sedating doses to reduce aggression in Alzheimer's patients.

While cholinesterase inhibitors appear to have a small but measurable effect on depression, dysphoria, apathy/indifference, and anxiety, these are not considered to be an effective short-term treatment for agitation or aggression. Memantine has shown some benefits in the treatment of Alzheimer's patients. However, a well-controlled randomized trial in Moderate to Severe Alzheimer's patients with clinically significant agitation did not demonstrate a reduction in agitation with memantine. Anticonvulsants, including valproic acid, have not shown positive results in recent studies. Anti-psychotics such as risperidone, while having some efficacy at managing agitation and aggression, carry an FDA black box warning for increased mortality in elderly patients with dementia and thus are contraindicated in this patient population.

There is therefore a need for new and improved methods for treating and managing aggression and agitation in a variety of patient groups, including in Alzheimer's disease and other dementias.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a person skilled in the art.

### SUMMARY OF THE INVENTION

In a first aspect, the present invention provides a method of treating or preventing aggression, agitation, irritability and/or anger in a subject, comprising administering to a subject in need thereof, a therapeutically effective amount of a compound of Formula (I)

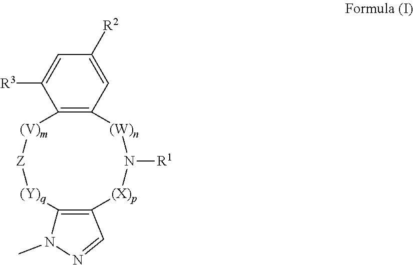

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅alkyl;
R³ is selected from: H, OH, halogen, an optionally substituted C₁₋₅ alkyl or an optionally substituted OC₁₋₅alkyl;
R⁴ is an optionally substituted C₁₋₅ alkyl;
m is 0 or 1;
n is 0 or 1;
p is 0 or 1; and
q is 0 or 1;
or a pharmaceutically acceptable salt or prodrug thereof, thereby treating the aggression, agitation, irritability and/or anger in the subject.

In another aspect, there is provided a use of a compound of formula (I) or a pharmaceutically acceptable salt or prodrug thereof, in the manufacture of a medicament for treating or preventing aggression, agitation, irritability and/or anger in a subject.

Further still, the invention provides a compound of formula (I) or a pharmaceutically acceptable salt or prodrug thereof, for treating or preventing aggression, agitation, irritability and/or anger in a subject.

In a further aspect, the invention provides a compound of formula (I) or a pharmaceutically acceptable salt or prodrug thereof, for use in treating aggression, agitation, irritability and/or anger in a subject.

The present invention also provides a pharmaceutical composition comprising a compound of formula (I) or a pharmaceutically acceptable salt or prodrug thereof, for use in treating or preventing aggression, agitation, irritability and/or anger in a subject.

In any aspect, the aggression, agitation, irritability and/or anger may be associated with or caused by cognitive decline, a cognitive disorder, and/or an intellectual disability in the subject.

The cognitive decline, cognitive disorder or intellectual disability may be associated with or caused by physical change or injury to the brain. Alternatively, the cognitive disorder or intellectual disability may be associated with or caused by a neurodevelopmental disorder.

The physical change or injury to the brain may be the result of a neurodegenerative condition or may be an acquired brain injury resulting from one or more of: a chemical injury (e.g., alcohol or drug-induced dementia and associated neurodegenerative conditions), compression or blunt trauma to the brain leading to traumatic brain injury (TBI), ischemic injury resulting from a transient ischemic attack (TIA), ischemic stroke or haemorrhagic stroke, or injury resulting from an infection.

In any aspect, the neurodegenerative condition may be characterised by the presence of abnormal protein deposits in the brain, including amyloidopathies, synucleinopathies or taupathies. The neurodegenerative condition or disorder may include Alzheimer's disease (AD), Lewy-bodies disease (Dementia with Lewy bodies (DLB)), Huntington's disease, Creutzfeldt-Jakob disease (CJD), Gaucher Disease Type 3, or Parkinson's disease. The neurodegenerative disease may be vascular dementia, frontotemporal dementia or other form of dementia not typically associated with deposition of abnormal protein deposits.

In any aspect of the invention, the aggression, agitation, irritability and/or anger may be associated with one or more of AD, vascular dementia, frontotemporal dementia, mixed dementia or Korsakoff syndrome.

Preferably, the aggression, agitation, irritability and/or anger is associated with AD. Accordingly, the present invention provides a method for treating or preventing aggression, agitation, irritability and/or anger associated with Alzheimer's disease (AD), the method comprising administering to a subject diagnosed with or suspected of having AD, a therapeutically effective amount of a compound of Formula (I), or a pharmaceutically acceptable salt or prodrug thereof, thereby treating or preventing the aggression, agitation, irritability and/or anger associated with Alzheimer's disease (AD) in the subject.

The neurodevelopmental disorder may be any neurodevelopmental disorder associated with or caused by a genetic mutation or genetic risk variants (risk variants). The genetic mutation or risk variants causing or associated with the neurodevelopmental disorder, may be a single gene mutation or risk variant, or the neurodevelopmental disorder may have a polygenetic cause. The single gene mutation or risk variant may include: a mutation in one or more of the genes listed in the SFARI database or other predicted or known neurodevelopmental disorder susceptibility genes. In any embodiment, the mutation or risk variant may be in one or more of the genes: neuroligin-3, neuroligin-4, ARID1B (AT-rich interaction domain 1B) ASH1L (ASH1 like histone lysine methyltransferase), CHD2 (chromodomain helicase DNA binding protein 2), CHD8 (chromodomain helicase DNA binding protein 8), DYRK1A (dual specificity tyrosine phosphorylation regulated kinase 1A), POGZ (pogo transposable element derived with ZNF domain), SHANK3 (SH3 and multiple ankyrin repeat domains 3), SYNGAP1 (synaptic Ras GTPase activating protein 1), methyl CpG binding protein 2 (MECP2), FMR1 (FMRP translational regulator 1), ACTL6B, ACY1, AHI1, ALDH1A3, ANKS1B, AP1S2, ARID2, ATP1A1, ATP1A3, ALG6, ANKRD17, BCORL1, BRWD3, C12orf57, CACNA1A, CAMK2A, CAMK2B, CONK, CDK13, CDK8, CEP290, CHD1, CDK19, CHKB, CLCN4, CNKSR2, CNTNAP2, CSNK2A1, CSNK2B, CTNNA2, CUX2, CYP27A1, DEPDCS, DHX30, DDX23, DLL1, DMD, DOLK, EEF1A2, ELP2, FBXO11, FBRSL1, FGF13, FRMPD4, GABBR2, GABRA3, GATM, GALNT2, HCFC1, HCN1, HDAC4, HDAC8, HEPACAM, HERC2, HNRNPD, HNRNPK, HNRNPR, HOXA1, HUWE1, INTS1, KAT6A, HNRNPUL2, KIFSC, KIF1A, KPTN, LNPK, MED12L, NACC1, NFIB, NFIX, NR2F1, NTNG1, NTNG2, NTRK2, OCRL, PACS2, PAK1, PAX6, NOVA2, PCCA, PCCB, NSD2, PHF8, PIK3R2, POU3F3, PPM1D, PPP2CA, PRKD1, PRODH, PRR12, POLR3A, PPP3CA, RAC1, RAD21, RALA, RFX4, RHEB, RLIM, RNF135, RORA, RPS6KA3, SATB2, RFX7, RIMS2, RSRC1, SETDIB, SGSH, SETD1A, SIK1, SLC1A2, SIN3B, SLC45A1, SMARCA2, SMC1A, SMC3, SNX14, STAG1, SOX6, SPTBN1, SYNE1, SYT1, TAF1, TBC1D23, TBX1, SUPT16H, TET3, TM4SF20, TRAF7, TRAPPC6B, TRRAP, TTI2, TTN, UNC13A, USP7, USP9X, VAMP2, WASF1, WDR26, XPC, YWHAG, ZMIZ1, ZMYM2, ZSWIM6, GNB2, ZBTB7A, KCNA2, TFE3, H1-4, PTPN4, and GRIA3. In certain embodiments, the genetic mutation may include the R451C point mutation in the neuroligin-3-gene.

In any embodiment, the neurodevelopmental disorder associated with or caused by a genetic mutation or risk variant may be selected from the group consisting of: Fragile-X Syndrome, X-Linked Intellectual Disability-Hypotonia-Facial Dysmorphism-Aggressive Behaviour Syndrome, Kleefstra Syndrome, Hunters Syndrome (MPS II), ADNP (activity dependent neuroprotector homeobox) syndrome, Rett syndrome, Autism Spectrum Disorders, Prader-Willi syndrome, Angelman Syndrome, Brunner syndrome, Cri du Chat syndrome, Cornelia de Lange syndrome, Smith-Lemli-Opitz syndrome, Smith-Magenis syndrome, Tuberous Sclerosis Complex, CHARGE syndrome, sotos syndrome, attention-deficit/hyperactivity disorder (ADHD) and PTEN associated disorders.

The neurodevelopmental disorder may also be the result of a congenital brain injury, including a physical or chemical injury (such as alcohol or drugs) or injury due to inflammatory immune responses or metabolic disturbances. The neurodevelopmental disorder may be cerebral palsy, fetal alcohol spectrum disorder or attention-deficit/hyperactivity disorder (ADHD).

In certain preferred embodiments, the neurodevelopmental disorder is ADHD.

In preferred embodiments of the invention the therapeutically effective amount of the compound of Formula (I), or a pharmaceutically acceptable salt or prodrug thereof, does not simultaneously cause pronounced sedation of the subject receiving treatment.

In any method or use of the invention, the compound of Formula (I), or a pharmaceutically acceptable salt or prodrug thereof, is administered orally, intranasally, systemically (e.g., subcutaneously, intramuscularly, intraperitoneally, intravenously) or rectally.

The present invention also provides a kit comprising an effective amount of a compound of Formula (I) or a pharmaceutically acceptable salt and/or prodrug thereof, optionally, wherein the kit includes written instructions for performing the methods of the invention. In some embodiments, these pharmaceutical compositions and kits may be used in any of the methods described herein.

In any aspect of the invention, including in any method, use or kit of the invention, the compound of Formula (I) is:

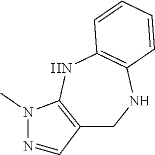

or a pharmaceutically acceptable salt or prodrug thereof.

The compound may be a hydrochloride salt of Compound 1, such as the di-hydrochloride salt (CMPD1-2HCL).

The compound may be a phosphoric acid addition salt of Compound 1 (CMPD1-PO4). The phosphoric acid addition salt may be referred to as a phosphate salt of Compound 1.

As used herein, except where the context requires otherwise, the term “comprise” and variations of the term, such as “comprising”, “comprises” and “comprised”, are not intended to exclude further additives, components, integers or steps.

It must be noted that as used herein and in the appended claims, the singular forms “a”, “an” and “the” include plural reference unless the context clearly dictates otherwise. Thus, for example, a reference to “a symptom” and/or “at least one symptom” may include one or more symptoms, and so forth.

The term “and/or” can mean “and” or “or”.

The term “(s)” following a noun contemplates the singular or plural form, or both.

Unless otherwise herein defined, chemical terms will have their general meanings known in the art.

As used herein, the term “C₁₋₅ alkyl” either used alone or in compound terms, refers to monovalent straight chain or branched hydrocarbon groups, having 1 to 5 carbon atoms. As understood by a person skilled in the art, the term “C₁₋₅ alkyl” means an alkyl chain with 1, 2, 3, 4 or 5 carbon atoms or a range comprising any of two of those integers including 1-2, 1-3, 1-4, 1-5, 2-3, 2-4, 2-5, 3-4, 3-5 and 4-5. Suitable alkyl groups include, but are not limited to: methyl, ethyl, propyl, isopropyl, n-butyl, sec-butyl, tert-butyl, n-pentyl, neopentyl, iso-pentyl and tert-pentyl. The C₁₋₄alkyl may be optionally substituted with one or more substituents. The substituents may be in any position of the carbon chain. Suitable substituents include, but are not limited to: OH, NH₂, halogen, NH(C₁₋₅alkyl), N(C₁₋₅ alkyl)₂, CN, NO₂, CO₂H, or OC₁₋₅alkyl.

The terms “hydroxy” and “hydroxyl” refer to the group —OH.

As used herein, the term “OC₁₋₅ alkyl” either used alone or in compound terms, refers to alkoxy groups having 1 to 5 carbon atoms. As understood by a person skilled in the art, the term “OC₁₋₅alkyl” means an alkoxy group with 1, 2, 3, 4 or 5 carbon atoms or a range comprising any of two of those integers and including 1-2, 1-3, 1-4, 1-5, 2-3, 2-4, 2-5, 3-4, 3-5 and 4-5. Suitable OC₁₋₅alkyl groups include, but are not limited to, methoxy, ethoxy, propyloxy, isopropyloxy, n-butyloxy, sec-butyloxy, tert-butyloxy, n-pentyloxy, neopentyloxy, iso-pentyloxy and tert-pentyloxy. The OC₁₋₅alkyl may be optionally substituted with one or more substituents. The substituents may be in any position of the carbon chain. Suitable substituents include, but are not limited to: OH, NH₂, halogen, NH(C₁₋₅alkyl), N(C₁₋₅ alkyl)₂, CN, NO₂, CO₂H, or OC₁₋₅alkyl.

As used herein, the term “halo” or “halogen” refers to fluorine (fluoro), chlorine (chloro), bromine (bromo) or iodine (iodo).

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. KNX100 dose response. Vehicle treated single housed mice spent significantly more time fighting (A), huddling (B) and travelled significantly more distance (D) than group housed mice. There was no difference in grooming between group housed and vehicle treated single housed mice (D). KNX100 (dihydrochloride salt thereof) dose dependently inhibited fighting (A) and distance travelled (D) and dose dependently increased huddling (B) and grooming (C). # p≤0.05, # # p≤0.05 vs group housed. ***** p≤0.00001 linear trend contrast for dose (trend contrast assessed single-housed groups only).

FIG. 2. Oral dosing of KNX100. Mice were divided into three groups: VEH (group housed), VEH (single housed) and KNX100 (single housed, 20 mg/kg PO). Mice received either vehicle saline or KNX100 (dihydrochloride salt thereof) in saline via gavage 30 min prior to a 5 min aggression test session. Single housed mice treated with vehicle spent significantly longer fighting than vehicle treated group housed mouse. KNX100 treated mice spent significantly less time fighting than the single housed vehicle treated mice, and did not differ significantly in time spent fighting relative to the group housed mice.

FIG. 3. KNX100 vs risperidone. Vehicle treated single housed mice spent significantly more time huddling than group housed mice (B). There was no difference in time spent fighting (A) or grooming (C) or in distance travelled (D) between group housed and vehicle treated single housed mice. Both 0.1 mg/kg risperidone and 10 mg/kg KNX100 (dihydrochloride salt thereof) significantly inhibited fighting (A) and increased huddling (B). Only risperidone significantly increased time spent grooming (C). Both risperidone and KNX100 significantly reduced distance travelled relative to vehicle treated single housed mice; however, risperidone caused significantly more inhibition of locomotor activity than KNX100 (D). # p≤0.05, # # p≤0.01, # # # # p≤0.0001 vs group housed. ** p≤0.01, *** p≤0.001, ***** p≤0.00001 vs vehicle treated single housed. + + + p≤0.001 vs 0.1 mg/kg risperidone.

FIG. 4. Effects of KNX100 phosphate salt on frequency of attacks of the intruder mouse. Data are presented as mean±SEM. *p<0.05, compared to vehicle-treated APP/PS1 group (Dunnett's test).

FIG. 5. Effects of KNX100 phosphate salt on latency to attack the intruder mouse. Data are presented as mean±SEM. *p<0.05, compared to vehicle-treated APP/PS1 mice (Dunnett's test). ˜p<0.086, compared to vehicle-treated APP/PS1 mice (Dunnett's test) and p=0.022 using Fisher's LSD test.

FIG. 6. Effects of acute vs subchronic KNX100 on fighting in isolated mice. Acute and subchronic KNX100 (phosphate salt thereof; 15 mg/kg freebase equivalent p.o.) both prevented the increased time spent fighting (A), greater number of fights (B), and reduced latency to first fight (C) in isolated mice and were equally efficacious. Bars and error bars (A & B) represent Mean+SEM. Open circles are the raw data. ***p<0.001 vs VEH group housed, ###p<0.001 vs VEH single housed.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All these different combinations constitute various alternative aspects of the invention.

For purposes of interpreting this specification, terms used in the singular will also include the plural and vice versa.

There are currently no approved medications which specifically target the reduction of aggression, agitation, irritability, and/or anger in neurodegenerative diseases such as Alzheimer's disease and dementia. To date, attempts to identify a serenic drug which can safely treat these conditions in these particular patient groups have been unsuccessful. Attempts to re-purpose drugs known for their ability to promote social interactions have been unsuccessful and studies therefore indicate that the ability of a drug to promote prosocial behaviours is not a reliable indicator of the utility of that same drug to treat aggression. For example, benzodiazepines that increase social interaction in rodents increase aggression in models of maladaptive aggression.

Against this background, the present inventors have surprisingly determined that compounds of Formula (I) (shown below) have the unexpected property of being able to significantly inhibit aggression and agitation in models used to identify candidate drugs for use in reducing aggression in various conditions, including in a model of neurodegenerative disease. While compounds of Formula (I) had previously been described to have powerful anti-addictive effects in preclinical models, it was surprisingly found that that these compounds can also have a profound effect on aggressive behaviour.

Accordingly, the present invention provides a method of treating or preventing aggression, agitation, irritability and/or anger in a subject, the method including a step of administering to the subject a compound of Formula (I), or a pharmaceutically acceptable salt, tautomer, N-oxide, solvate, polymorph and/or prodrug thereof.

The present invention also relates to a use of a compound of Formula (I), or a pharmaceutically acceptable salt, tautomer, N-oxide, solvate, polymorph and/or prodrug thereof, in the manufacture of a medicament for treating or preventing aggression, agitation, irritability and/or anger in a subject.

The present invention also relates to a compound of Formula (I), or a pharmaceutically acceptable salt, tautomer, N-oxide, solvate, polymorph and/or prodrug thereof, for use in a method for treating or preventing aggression, agitation, irritability and/or anger in a subject.

### Conditions and Subjects to be Treated

The methods, compositions and uses of the invention find particular application in the treatment and/or prevention of aggression, agitation, irritability and/or anger in a variety of patient groups, especially in patients having cognitive decline, a cognitive disorder, and/or an intellectual disability, including those caused by or associated with one or more of:
physical change or injury to the brain;
   including neurodegenerative diseases characterised by abnormal protein deposits in the brain, neuronal loss;
   dementia due to infection;
   traumatic brain injury;
   ischemic brain injury (e.g., following haemorragic stroke, transient ischemic attack or ischemic stroke);
   chemical-induced injury to the brain (such as alcohol-induced dementia or injury caused by exposure to drugs or heavy metals);
   nutrient deficiency-induced injury to the brain (such as Korsakoff syndrome caused by severe thiamine deficiency resulting from alcohol misuse or other causes);
   neurodevelopmental disorders (especially those associated with a genetic mutation or risk variant, but also including those associated or caused by chemical or physical injury to the brain during early development).

As used herein, “aggression” refers to an overt hostile and harmful behaviour directed to oneself, toward others and/or toward objects. Aggression may be verbal, non-verbal or physical. It can be classified as adaptive aggression or maladaptive aggression.

The aggression, agitation, irritability and/or anger requiring treatment in accordance with the present invention, may be associated with cognitive decline that is the result of a neurodegenerative condition. The neurodegenerative condition may be selected from: amyloidopathies, synucleinopathies or taupathies. In any embodiment, the neurodegenerative disease may include Alzheimer's disease, frontotemporal dementia, Huntington's disease, Parkinson's disease, mild cognitive impairment, Lewy-bodies disease (Dementia with Lewy bodies (DLB)), or Creutzfeldt-Jakob disease (CJD).

The aggression, agitation, irritability and/or anger may be the result of a blunt trauma to the brain, leading to traumatic brain injury (TBI).

The aggression, agitation, irritability and/or anger may be associated with ischemic brain injury, which may be the result of ischemia, such as a transient ischemic attack or stroke.

In any aspect, the aggression, agitation, irritability and/or anger may be idiopathic i.e., with no known cause.

As used herein, cognitive disorder, or neurocognitive disorder (NCD) refers to a mental health and/or neurological disorder that primarily affect cognitive abilities including learning, memory, perception, and/or problem solving. Neurocognitive disorders include delirium and mild and major neurocognitive disorder (previously known as dementia). They are defined by deficits in cognitive ability that are acquired (as opposed to developmental), typically represent decline, and may have an underlying brain pathology. The DSM-5 defines six key domains of cognitive function: executive function, learning and memory, perceptual-motor function, language, complex attention, and social cognition.

Although Alzheimer's disease accounts for the majority of cases of neurocognitive disorders, there are various medical conditions that affect mental functions such as memory, thinking, and the ability to reason, including frontotemporal degeneration, vascular dementia, Huntington's disease, Lewy body disease, traumatic brain injury (TBI), Parkinson's disease, prion disease, and dementia/neurocognitive issues due to HIV infection.

Neurocognitive disorders are diagnosed as mild and major based on the severity of their symptoms. The skilled person will be familiar with standard approaches to identifying subjects at risk of dementia, or related neurocognitive disorder, or diagnosing a subject with a neurodegenerative disease, including any of those described herein.

Similarly, the skilled person will be familiar with standard methods for identifying whether a patient has an intellectual disability, e.g., associated with a neurodevelopmental disorder; and furthermore determining whether the patient has aggression, agitation, irritability and/or anger requiring treatment in accordance with the invention. Such methods may include genetic testing, obtaining a complete medical and family medical history, and various behavioural and cognitive tests.

In the context of neurodegenerative disorders, for example, while there is no definitive test to diagnose many of these conditions (including AD) in a patient that is still living, the skilled person will be familiar with the information gathering required in order to make a prediction of likely diagnosis and thereby identify a patient for treatment in accordance with the present invention. For example, the diagnosis of AD typically includes gathering the following information:
complete medical and family medical history;
basic medical tests (blood, urine tests);
neuropsychological and intellectual functioning tests (e.g., memory tests, problem-solving, language tests, mini-mental state examination (MMSE));
interviews with family members to develop a comprehensive picture of the behaviours of the patient;
medical imaging of the brain (e.g., MRI, CT, SPECT or PET scans) to identify areas containing plaques to exclude other cerebral pathology and subtypes of dementia and to predict conversion from prodromal stages (mild cognitive impairment) to AD;
genetic factors (family history of AD);
environmental factors (exposure to aluminium, zinc and other metals).

The National Institute of Neurological and Communicative Disorders and Stroke (NINCDS) and the Alzheimer's Disease and Related Disorders Association (ADRDA, now known as the Alzheimer's Association) established the most commonly used NINCDS-ADRDA Alzheimer's Criteria for diagnosis in 1984, extensively updated in 2007. These criteria require that the presence of cognitive impairment, and a suspected dementia syndrome, be confirmed by neuropsychological testing for a clinical diagnosis of possible or probable AD. A histopathologic confirmation including a microscopic examination of brain tissue is required for a definitive diagnosis. Good statistical reliability and validity have been shown between the diagnostic criteria and definitive histopathological confirmation. Eight intellectual domains are most commonly impaired in AD—memory, language, perceptual skills, attention, motor skills, orientation, problem solving and executive functional abilities. These domains are equivalent to the NINCDS-ADRDA Alzheimer's Criteria as listed in the Diagnostic and Statistical Manual of Mental Disorders (DSM-IV-TR) published by the American Psychiatric Association.

AD advances in stages, ranging from mild forgetfulness to severe dementia. The course of the disease and the rate of decline varies from person to person. The duration from onset of symptoms to death can be from 5 to 20 years.

The early signs of dementia are subtle and vague and may not be immediately obvious. Some common symptoms may include:
progressive and frequent memory loss
confusion
personality change
apathy and withdrawal
loss of ability to perform everyday tasks.

The first symptoms of AD (pre-dementia) are often mistakenly attributed to ageing or stress. Detailed neuropsychological testing can reveal mild cognitive difficulties up to eight years before a person fulfils the clinical criteria for diagnosis of AD. These early symptoms can affect the most complex activities of daily living. The most noticeable deficit is short term memory loss, which shows up as difficulty in remembering recently learned facts and inability to acquire new information.

Subtle problems with the executive functions of attentiveness, planning, flexibility, and abstract thinking, or impairments in semantic memory (memory of meanings, and concept relationships) can also be symptomatic of the early stages of AD. Apathy can be observed at this stage, and remains the most persistent neuropsychiatric symptom throughout the course of the disease. Depressive symptoms, irritability and reduced awareness of subtle memory difficulties are also common. The preclinical stage of the disease has also been termed mild cognitive impairment (MCI). This is often found to be a transitional stage between normal ageing and dementia. MCI can present with a variety of symptoms, and when memory loss is the predominant symptom, it is termed “amnestic MCI” and is frequently seen as a prodromal stage of Alzheimer's disease.

In people with AD, the increasing impairment of learning and memory eventually leads to a definitive diagnosis. In a small percentage, difficulties with language, executive functions, perception (agnosia), or execution of movements (apraxia) are more prominent than memory problems. AD does not affect all memory capacities equally. Older memories of the person's life (episodic memory), facts learned (semantic memory), and implicit memory (the memory of the body on how to do things, such as using a fork to eat or how to drink from a glass) are affected to a lesser degree than new facts or memories.

Language problems are mainly characterised by a shrinking vocabulary and decreased word fluency, leading to a general impoverishment of oral and written language. In this early stage, the person with Alzheimer's is usually capable of communicating basic ideas adequately. While performing fine motor tasks such as writing, drawing or dressing, certain movement coordination and planning difficulties (apraxia) may be present, but they are commonly unnoticed. As the disease progresses, people with AD can often continue to perform many tasks independently, but may need assistance or supervision with the most cognitively demanding activities.

Progressive deterioration eventually hinders independence, with subjects being unable to perform most common activities of daily living. Speech difficulties become evident due to an inability to recall vocabulary, which leads to frequent incorrect word substitutions (paraphasias). Reading and writing skills are also progressively lost. Complex motor sequences become less coordinated as time passes and AD progresses, so the risk of falling increases. During this phase, memory problems worsen, and the person may fail to recognise close relatives. Long-term memory, which was previously intact, becomes impaired.

Behavioural and neuropsychiatric changes become more prevalent with progressing neurodegenerative/neurocognitive decline. Common manifestations are wandering, irritability and labile affect, leading to crying, outbursts of unpremeditated aggression, or resistance to caregiving. Sundowning can also appear. Approximately 30% of people with AD develop illusionary misidentifications and other delusional symptoms. Subjects also lose insight of their disease process and limitations (anosognosia). Urinary incontinence can develop. These symptoms create stress for relatives and carers, which can be reduced by moving the person from home care to other long-term care facilities

Dementia with Lewy bodies (DLB) is a dementia that has the primary symptoms of visual hallucinations and “Parkinsonism”. Parkinsonism is the symptoms of Parkinson's disease, which includes tremor, rigid muscles, and a face without emotion. The visual hallucinations in DLB are generally very vivid hallucinations of people or animals and they often occur when someone is about to fall asleep or just waking up. Other prominent symptoms include problems with attention, organization, problem solving and planning (executive function), and difficulty with visual-spatial function.

Although imaging studies do not necessarily make the diagnosis of DLB, some signs are particularly common. A person with DLB often shows occipital hypoperfusion on SPECT scan or occipital hypometabolism on a PET scan. Generally, a diagnosis of DLB is straightforward and unless it is complicated, a brain scan is not always necessary.

The skilled person will also be familiar with standard approaches for determining whether a subject has aggression, agitation, irritability and/or anger associated with a traumatic brain injury (TBI). The subject may have inflammation and/or Tau deposition in one or more brain regions, which can be identified by magnetic resonance imaging (MRI) and/or positron emission tomography (PET) imaging.

A complete medical history will also aid in the determination of whether the aggression, agitation, irritability and/or anger is associated with or likely caused by a TBI. The TBI may be caused by fall, an assault, a motor vehicle accident, a sport or recreational injury, shaken baby syndrome, a gunshot wound, a combat injury, or an electric shock.

The patient/subject may have one or more additional neurological symptoms associated with the TBI, including one or more of memory loss, depression, mood swings, balance problems, anxiety, substance abuse, obsessive compulsive disorder, and muted emotions.

The severity of a TBI can be estimated using one or more tests known in the art, such as but not limited to, Glasgow Coma Scale (GCS) score, measurements for level of TBI (e.g., ranking a person's level of consciousness, memory loss, and GCS), speech and language tests, cognition and neuropsychological tests, and imaging tests. See, www.nichd.nih.gov/health/topics/tbi/conditioninfo/diagnose. Methods and tests for the diagnosis of TBI are also described, e.g., in Byrnes et al., Front Neuroenergetics, 2013, 5:13; Mutch et al., Neurosurg Clin N Am, 2016, 27:409-439; Prince et al., Brain Sci, 2017, 7:105; and Reis et al., Int J Mol Sci, 2015, 16:11903-11965.

Similarly, methods for determining the presence of ischemic brain injury, (e.g., following haemorragic stroke, transient ischemic attack or ischemic stroke), chemical-induced brain injury, nutrient-deficiency associated brain injury and their likely association with aggression, agitation, irritability and/or anger will be familiar to the person skilled in the art.

In some embodiments, a subject to be treated is identified using one or more cognition and neuropsychological tests. Cognition and neuropsychological tests include, but are not limited to, tests that assess the subject's cognitive (e.g., thinking, reasoning, problem solving, information processing, and memory functions), language, behavioral, motor, and executive functions.

A subject may be identified for treatment in accordance with the methods of the present invention by assessment of agitation or aggression based on established psychiatric assessments known to the skilled person. A variety of suitable scales for identifying aggression and agitation are known and it will be understood that the invention is not limited by which scale is used. In certain embodiments, a subject requiring treatment may be identified by utilising one or more of:
the Neuropsychiatric Inventory-Clinician Agitation and Aggression rating scale (NPI-C A+A);
the Neuropsychiatric Inventory-Nursing Home Version (NPI-NH) agitation/aggression scale;
the Cohen-Mansfield Agitation Inventory (CMAI) total score or the Cohen-Mansfield Agitation Inventory-Short Form (CMAI-SF) total score;
a method that evaluates caregiver distress (for example, NPI-C caregiver distress),
a clinician's global assessment of agitation, (for example, the Alzheimer's Disease Cooperative Study—Clinical Global Impression of Change or mADCS-CGIC), neuropsychiatric inventory questionnaire (NPI-Q) and/or cognitive status (MMSE);
Columbia University for Psychopathology in Alzheimer's disease (CUPSAD);
Positive and Negative Syndrome Scale (PANSS);
the BEHAVE-AD rating scale;
the Multi-Dimensional Dementia Assessment Scale (MDDAS);
the Agitation Calmness Evaluation Scale; or
the Behavioural Activity Rating Scale.

The scale utilised may be a composite scale that does not separate aggression from agitation. Alternatively, the scale used may enable separate classification of aggressive or agitated behaviours.

The “Neuropsychiatric Inventory” or “NPI” refers to the 12-item NPI (Cummings J L, et al, Neurology. 1994; 44(12):2308-14) which assesses psychopathology in dementia patients and has been widely used in AD clinical trials. It evaluates 12 neuropsychiatric symptoms (NPS) or domains common in dementia. Neuropsychiatric symptoms are rated on the basis of questions administered to the subject's caregiver/study partner. The score for each NPI domain is the frequency score (0-4) multiplied by the severity score (0-3), based on the item/question with the highest score or severity. The maximal domain score is 12 (4×3), and the possible values for each domain include 1, 2, 3, 4, 6, 8, 9, and 12. If all symptoms of a domain are absent, then the total domain score is 0. The maximal score on NPI-A/A (NPI Agitation and NPI Aggression) is 12 (4 for frequency, 3 for severity). Thus, “NPI-A/A” reflects the rating of the combined NPI Agitation and Aggression domains. The NPI domains of irritability, aberrant motor behaviour, and delusions also occur frequently in AD patients with agitation and aggression. The maximal score on each is 12 (4 for frequency, 3 for severity). The total NPI score represents overall burden of NPS in AD patients. This is calculated by summing the domain scores from all 12 domains. The maximal total score for the 12-item NPI is 144 (12×12). Higher scores on the NPI are associated with greater frequency and severity of NPS; therefore, a negative change from baseline score indicates an improvement from baseline.

“Neuropsychiatric Inventory-Clinician rating scale” or “NPI-C” is an expanded form of the widely used Neuropsychiatric Inventory (12-item NPI, Cummings 1997). “Neuropsychiatric Inventory-Clinician Agitation and Aggression” or “NPI-C A+A” reflects the NPI-C rating of the combined agitation and aggression domains. The NPI-C was developed and validated by a consortium of neuropsychiatric AD experts. The NPI-C scale has the following characteristics that support its use as a primary outcome measure for clinical trials of agitation and aggression in AD. These characteristics include: (a) having separate domains to evaluate agitation and aggression (NPI-C Agitation and NPI-C Aggression), (b) increasing the number of questions/items within each of these two domains to provide a comprehensive profile of these behaviours (NPI-C A+A has a total of 21 items versus 8 items on the NPI-A/A), and (c) utilizing an experienced and trained rater to perform the assessments of clinical severity after direct patient observation and interviews of both the patient and caregiver. The NPI-C scoring is based on the clinician's rating of the severity of each NPS or domain (i.e., agitation or aggression) after an interview of both patient and their caregiver/study partner. Each domain score of the NPI-C is calculated by summing the “clinical severity” scores of all items/questions within that domain. For each domain, the overall clinical severity score ranges 0 to 3 for each item/question; therefore, the maximal score for each domain is 3×number of items within that domain. Therefore, the maximal score for agitation is 39 (13×3) and for aggression is 24 (8×3). The range on the NPI-C combined Agitation and Aggression (NPI-C A+A) scores is 0-63. Higher scores on the NPI-C domains are associated with a greater clinical severity of symptoms, therefore a negative change from baseline score indicates an improvement from baseline.

The NPI-Q is a brief questionnaire form of the NPI developed for use in routine clinical practice (NPI-Q) (see, Kaufer, D I, J Neuropsychiatry Clin Neurosci. 2000 Spring;

12(2):233-9). The NPI-NH was developed to assess psychopathology in patients with dementia in nursing homes and evaluates. The nursing home version of this scale was designed to examine psychopathology in nursing home patients and has been validated for use in this population (Wood et al., Am. J. Geriatr. Psychiatr. 2000, vol. 8(1), 75-83.)

The NPI-NH includes ten behavioural domains and two neurovegetative changes domains. The behavioural domains are delusions (Domain A), hallucinations (Domain B), agitation/aggression (Domain C), depression/dysphoria (Domain D), anxiety (Domain E), elation/euphoria (Domain F), apathy/indifference (Domain G), disinhibition (Domain H), irritability/lability (Domain I), and aberrant motor behaviour (Domain J). The neurovegetative changes domains are sleep and night-time behaviour disorders (Domain K) and appetite and eating disorders (Domain L). (Wood et al., Am. J. Geriatr. Psychiatr 2000, vol. 8(1), 75-83).

The NPI-NH is based on responses from an informed professional caregiver involved in the daily care of the resident. The interview is best conducted in the absence of the resident to facilitate an open discussion of behaviours that may be difficult to describe with the resident present.

To determine the score for a given domain, the caregiver rates frequency and severity of the patient's behaviour for that particular domain. The frequency is rated on a 0-4 point scale. For example, the frequency is rated as a) 0 for not present; b) 1 for occasional; c) 2 for often; d) 3 for frequent; and e) 4 for very frequent occurrence of the behaviour in question. The severity is rated on a 1-3 point scale. For example, the severity is rated as a) 1 for mild; b) 2 for moderate; and 3 for severe. The score of each domain is determined by multiplying the frequency rating score by the severity rating score. The score of each domain ranges from 0 to 12.

A total NPI-NH score can be calculated by adding all of the ten behavioural domain scores together.

The CMAI is a 14- or 28-item instrument assessing frequency of manifestations of agitation in the elderly based on directly observable behaviours (Werner et al., Geriatric Nursing, 1994, vol. 15(3), 142-146; Koss et al., Alzheimer's Disease and Associated Disorders, 1997, vol. 11, 45-50) including physically aggressive behaviours and verbally aggressive behaviours. (Cohen-Mansfield et al., J of Gerontology Med. Sci., 1989, vol. 44(3), M77-M84; Werner et al., Geriatric Nursing, 1994, vol. 15(3), 142-146; Koss et al., Alzheimer's Disease and Associated Disorders, 1997, vol. 11, 45-50.)

In any embodiment, the subject requiring treatment has a baseline score of 3 or greater, 4 or greater, 5 or greater, 6 or greater, 7 or greater, 8 or greater, 9 or greater, 10 or greater, 11 or greater, 12 or greater, 13 or greater, 14 or greater, 15 or greater, or 16 or greater in the psychosis sub scale (Domains A and B) of the NPI-NH. In another embodiment, the subject has a baseline score of 6 or greater in the psychosis subscale (Domains A and B) of the NPI-NH. In yet another embodiment, the subject has a baseline score of 9 or greater in the psychosis subscale (Domains A and B) of the NPI-NH. In yet another embodiment, the subject has a baseline score of 12 or greater in the psychosis subscale (Domains A and B) of the NPI-NH.

In any embodiment, the subject has a baseline score of 3 or greater, 4 or greater, 5 or greater, 6 or greater, 7 or greater, or 8 or greater in the agitation and aggression subscale (Domain C) of the NPI-NH. In another embodiment, the subject has a baseline score of 3 or greater, 4 or greater, 5 or greater, 6 or greater, 7 or greater, or 8 or greater in the depression/dysphoria subscale (Domain D) of the NPI-NH. In yet another embodiment, the subject has a baseline score of 3 or greater, 4 or greater, 5 or greater, 6 or greater, 7 or greater, or 8 or greater in the anxiety subscale (Domain E) of the NPI-NH. In yet another embodiment, the subject has a baseline score of 3 or greater, 4 or greater, 5 or greater, 6 or greater, 7 or greater, or 8 or greater in the elation/euphoria subscale (Domain F) of the NPI-NH. In yet another embodiment, the subject has a baseline score of 3 or greater, 4 or greater, 5 or greater, 6 or greater, 7 or greater, or 8 or greater in the apathy/indifference subscale (Domain G) of the NPI-NH. In yet another embodiment, the subject has a baseline score of 3 or greater, 4 or greater, 5 or greater, 6 or greater, 7 or greater, or 8 or greater in the disinhibition subscale (Domain H) of the NPI-NH. In yet another embodiment, the subject has a baseline score of 3 or greater, 4 or greater, 5 or greater, 6 or greater, 7 or greater, or 8 or greater in the disinhibition subscale (Domain H) of the NPI-NH. In yet another embodiment, the subject has a baseline score of 3 or greater, 4 or greater, 5 or greater, 6 or greater, 7 or greater, or 8 or greater in the irritability/lability subscale (Domain I) of the NPI-NH. In yet another embodiment, the subject has a baseline score of 3 or greater, 4 or greater, 5 or greater, 6 or greater, 7 or greater, or 8 or greater in the sleep and night-time behaviour disorders subscale (Domain J) of the NPI-NH. In yet another embodiment, the subject has a baseline score of 3 or greater, 4 or greater, 5 or greater, 6 or greater, 7 or greater, or 8 or greater in the sleep and night-time behaviour disorders subscale (Domain K) of the NPI-NH. In yet another embodiment, the subject has a baseline score of 3 or greater, 4 or greater, 5 or greater, 6 or greater, 7 or greater, or 8 or greater in the appetite and eating disorders subscale (Domain L) of the NPI-NH.

In any embodiment, the subject has a baseline score of 6 or greater in the psychosis sub scale (Domains A and B) of the NPI-NH; and/or has a baseline score of 3 or greater in the agitation and aggression subscale (Domain C) of the NPI-NH. In another embodiment, the subject has a baseline score of 6 or greater in the psychosis subscale (Domains A and B) of the NPI-NH; and/or has a baseline score of 6 or greater in the agitation and aggression sub scale (Domain C) of the NPI-NH. In yet another embodiment, the subject has a baseline score of 6 or greater in the psychosis subscale (Domains A and B) of the NPI-NH; and/or has a baseline score of 9 or greater in the agitation and aggression subscale (Domain C) of the NPI-NH. In yet another embodiment, the subject has a baseline score of 9 or greater in the psychosis subscale (Domains A and B) of the NPI-NH; and/or has a baseline score of 3 or greater in the agitation and aggression subscale (Domain C) of the NPI-NH. In yet another embodiment, the subject has a baseline score of 9 or greater in the psychosis subscale (Domains A and B) of the NPI-NH; and/or has a baseline score of 6 or greater in the agitation and aggression subscale (Domain C) of the NPI-NH. In yet another embodiment, the subject has a baseline score of 9 or greater in the psychosis subscale (Domains A and B) of the NPI-NH; and/or has a baseline score of 9 or greater in the agitation and aggression subscale (Domain C) of the NPI-NH. In yet another embodiment, the hu subject man has a baseline score of 12 or greater in the psychosis subscale (Domains A and B) of the NPI-NH; and/or has a baseline score of 3 or greater in the agitation and aggression subscale (Domain C) of the NPI-NH. In yet another embodiment, the subject has a baseline score of 12 or greater in the psychosis subscale (Domains A and B) of the NPI-NH; and/or has a baseline score of 6 or greater in the agitation and aggression subscale (Domain C) of the NPI-NH. In yet another embodiment, the subject has a baseline score of 12 or greater in the psychosis subscale (Domains A and B) of the NPI-NH; and/or has a baseline score of 9 or greater in the agitation and aggression subscale (Domain C) of the NPI-NH.

In an embodiment, the subject has no history of being prescribed for using an anti-psychotic agent. In another embodiment, the subject has a baseline score of 12 or greater in the psychosis subscale (Domains A and B) of the NPI-NH and/or has no history being prescribed for using an anti-psychotic agent. In yet another embodiment, the subject has a baseline score of 6 or greater in the agitation and aggression subscale (Domain C) of the NPI-NH and/or has no history of being prescribed for using an anti-psychotic agent. In yet another embodiment, the subject has a baseline score of 12 or greater in the psychosis sub scale (Domains A and B) of the NPI-NH; has a baseline score of 6 or greater in the agitation and aggression subscale (Domain C) of the NPI-NH; and/or has no history of being prescribed for using an anti-psychotic agent.

In certain embodiments of the invention, the methods of treatment may further comprise the steps of determining a baseline score on the NPI-NH for a human; and identifying a subject with a score of 6 or greater in the Agitation/Aggression subscale (Domain C) of the NPI-NH prior to administering a compound of Formula (I) to the subject.

Although examples of baseline and agitation and aggression scores are provided above in the context of the NPI-NH scale, it will be appreciate that any scale may be used for the purposes of identifying a subject in need of treatment according to the present invention; and for the purposes of determining treatment success. Such scales will be familiar to the skilled person and are also exemplified in the general summary of frequently used measurement scales for measurement of agitation and aggression in adult and aged patients provided in Volicer et al., (2017) CNS Spectrums: 1-8, incorporated herein by reference.

As used herein, treating aggression, agitation, irritability and/or anger in a subject in need thereof, shall be understood to refer to a reduction, amelioration or improvement in signs or symptoms of aggression, agitation, irritability and/or anger observed in the subject. The reduction, amelioration or improvement may include a reduced or less frequent incidence or occurrence of verbally agitated or verbally aggressive behaviour. The reduction, amelioration or improvement may include a reduced or less frequent incidence or occurrence of physically agitated or physically aggressive behaviour.

As used herein “preventing” or “prevention” is intended to refer to at least the reduction of likelihood of the risk of (or susceptibility to) developing aggression, agitation, irritability or other condition described herein (i.e., causing at least one of the clinical symptoms of the disease not to develop in a patient that may be exposed to or predisposed to the disease but does not yet experience or display symptoms of the disease). For example, prevention or preventative measures may be adopted for a patient deemed to be at risk, for example, a patient identified as having a dementia or other cognitive or neurodegenerative or other condition associated with aggression or agitation described herein. Preventative methods may also be used to prevent the occurrence of an aggressive or agitated state when a subject as identified herein, is placed in a situation or environment which is suspected to induce an aggressive or agitated response.

The terms “improvement,” “improved” and “improves” as used herein with respect to the clinical setting refer to a clinically relevant effect being achieved greater than about 5%, 10%, 20%, 30%, 40%, 50%, 60%, 70%, 80%, 90%, 100%, 110%, 120%, 130%, 140%, 150%, 160%, 170%, 180%, 200%, 300%, 400%, or 500% when compared to baseline after a specified period of time. In some embodiments, the improvement refers to improved efficacious effect in a single patient after the administration of a compound of Formula (I) as compared to baseline (i.e., prior to the administration of a compound of Formula (I)). In other embodiments, the improvement refers to the demonstration of efficacy by a greater percentage of patients demonstrating an efficacious effect after a specified period of time as compared to placebo or lack of treatment. In various embodiments, the percentage of patients demonstrating an efficacious effect is increased by greater than about 5%, 10%, 20%, 30%, 40%, 50%, 60%, 70%, 80%, 90%, 100%, 110%, 120%, 130%, 140%, 150%, 160%, 170%, 180%, 200%, 300%, 400%, or 500% when compared to placebo or lack of treatment. In some embodiments, the specified period of time is about two weeks, four weeks or six weeks. In one embodiment, the specified period of time is six weeks.

The terms “reduction,” “reduced” and “reduces” as used herein with respect to the clinical setting refer to a clinically relevant effect being achieved less than about 5%, 10%, 20%, 30%, 40%, 50%, 60%, 70%, 80%, 90%, 100%, 110%, 120%, 130%, 140%, 150%, 160%, 170%, 180%, 200%, 300%, 400%, or 500% when compared to baseline after a specified period of time or when compared to placebo or lack of treatment. In some embodiments, the specified period of time is about two weeks, four weeks or six weeks. In one embodiment, the specified period of time is six weeks.

In any embodiment or aspect of the invention, the agitated behaviour and/or aggressive behaviour may be one or more of the following: pacing and aimless wandering, such as constantly walking back and forth; inappropriate dressing or disrobing, such as putting on too many clothes, putting on clothing in a strange manner (e.g. putting pants on head), taking off clothing in public; spitting, such as spitting onto floor, and other people; cursing or verbal aggression, such as using words; swearing, use of obscenity, profanity, unkind speech or criticism, verbal anger, verbal combativeness; constant unwarranted request for attention or help, such as verbal or nonverbal unreasonable nagging, pleading, demanding; repetitive sentences or questions, such as repeating the same sentence or question one right after the other; hitting, such as physical abuse, striking others, pinching others, banging self/furniture; kicking, such as strike forcefully with feet at people or objects; grabbing onto people or things inappropriately, such as snatching, seizing roughly, taking firmly, or yanking; pushing, such as forcefully thrusting, shoving, moving putting pressure against; throwing things, such as hurl, violently tossing up in air, tipping off surfaces, flinging, intentionally spilling food; making strange noises, such as including crying, weeping, moaning, weird laughter, grinding teeth; screaming, such as loud shrill, shouting, piercing howl; biting, such as chomp, gnash, gnaw; scratching, such as clawing, scraping with fingernails; trying to get to a different place, such as trying to get out of the building, off the property, leaving inappropriately, trying to get into locked areas, trespassing within unit, into offices, other resident's room or closet; intentional falling, such as purposefully falling onto floor, include from wheelchair, chair, or bed; complaining—whining, complaining about self, somatic complaints, personal gripes or complaining about external things or other people; negativism, such as bad attitude, doesn't like anything, nothing is right; eating or drinking inappropriate substances, such as putting into mouth and trying to swallow items that are inappropriate; hurting self or other, such as burning self or other, cutting self or other, touching self or other with harmful objects; handling things inappropriately, such as picking up things that don't belong to them, rummaging through drawers, moving furniture, playing with food, faecal smearing; hiding things, such as putting objects under or behind something; hoarding things, such as putting many or inappropriate objects in purse or pockets, keeping too many of an item; tearing things or destroying property, such as shredding, ripping, breaking, stomping on something; performing repetitious mannerisms, such as stereotypic movement, such as patting, tapping, rocking self, fiddling with something, twiddling with something, rubbing self or object, sucking fingers, taking shoes on and off, picking at self, clothing, or objects, picking imaginary things out of air or off floor, manipulation of nearby objects in a repetitious manner; making verbal sexual advances, such as sexual propositions, sexual innuendo, or “dirty” talk; making physical sexual advances or exposing genitals, such as touching a person in an inappropriate sexual way, rubbing genital area, inappropriate masturbation, when not alone in own room or bathroom, unwanted fondling or kissing; and general restlessness—fidgeting, always moving around in seat, getting up and sitting down inability to sit still.

In certain embodiments, the verbally agitated behaviour and/or verbally aggressive behaviour includes, but is not limited to, shouting, screaming, swearing, making verbal sexual advances, cursing or verbal aggression, repetitive sentences or questions, making strange noises (weird laughter or crying), complaining, negativism, constant unwarranted request for attention or help, and making threats.

In certain embodiments, agitation may be understood to include excessive motor or verbal activity without any focus or intent, whereas aggression may be understood to include a provoked or unprovoked behaviour intended to cause harm. Aggression used in self-defence can be called “reactive aggression.”

Just as the skilled person will be familiar with methods for identifying a subject requiring treatment according to the present invention, the skilled person will also be able to determine the success of treatment. For example, the skilled person will be familiar with methods for assessing improvements in signs or symptoms of aggression or agitation in a subject according to any of the assessments described herein.

In some embodiments, an “improvement” may refer to a reduction of score on the scale or subscale of the Neuropsychiatric Inventory-Nursing Home Version (NPI-NH), the Alzheimer's Disease Cooperative Study-Clinical Global Impression of Change (ADSC-CGIC), the Cohen-Mansfield Agitation Inventory (CMAI) or the Cohen-Mansfield Agitation Inventory-Short Form (CMAI-SF). For example, an improvement refers to a reduction of a patient's total NPI-NH score from a score of 50 to a score of 40. In some embodiments, the improvement may optionally refer to one or more patients.

In certain embodiments, a successful treatment in accordance with the methods of the present invention may include one or more of:
an improved patient score in the Agitation/Aggression subscale of NPI-NH;
an improved patient score in the Sleep/Night-time Behaviour subscale of NPI-NH;
an improved patient score in the Cohen-Mansfield Agitation Inventory-Short Form (CMAI-SF) or Cohen-Mansfield Agitation Inventory (CMAI) total score;
an improved patient score in the aggressive behaviour subdomain of CMAI-SF or CMAI;
an improved patient score in the physically nonaggressive subdomain of CMAI-SF or CMAI;
an improved patient score in the verbally aggressive subdomain of CMAI-SF or CMAI.

In certain embodiments, the effect is determined by an improved score in the NPI-NH. In certain embodiments, the improved score in the NPI-NH is at least 2, at least 3, at least 4, at least 5, at least 6, at least 7, at least 8, or at least 9 points as compared to baseline score prior to administering a compound of Formula (I). In certain embodiments, the improved score in the NPI-NH is at least 2, at least 3, or at least 4 points as compared to baseline score prior to administering a compound of Formula (I).

In certain embodiments, the improved score in the psychosis subscale (Domains A and B) of the NPI-NH is at least 2, at least 3, at least 4, at least 5, at least 6, at least 7, at least 8, or at least 9 points as compared to baseline score prior to administering a compound of Formula (I). In certain embodiments, the improved score in the psychosis subscale (Domains A and B) of the NPI-NH is at least 2, at least 3, or at least 4 points as compared to baseline score prior to administering a compound of Formula (I).

In certain embodiments, the methods provided herein reduce the patient's NPI-NH score by at least 2, at least 3, at least 4, at least 5, at least 6, at least 7, at least 8, or at least 9 points as compared to baseline score prior to administering a compound of Formula (I). In certain embodiments, the methods provided herein reduce the patient's NPI-NH score by at least 2, at least 3, or at least 4 points as compared to baseline score prior to administering a compound of Formula (I).

In certain embodiments, the methods provided herein reduce the patient's score in the psychosis subscale (Domains A and B) of the NPI-NH by at least 2, at least 3, at least 4, at least 5, at least 6, at least 7, at least 8, or at least 9 points as compared to baseline score prior to administering a compound of Formula (I). In certain embodiments, the methods provided herein reduce the patient's score in the psychosis subscale (Domains A and B) of the NPI-NH by at least 2, at least 3, or at least 4 points as compared to baseline score prior to administering a compound of Formula (I).

One of ordinary skill in the art will understand that the improved score or the points of score reduced will be less than the baseline score.

In certain embodiments, the effect is determined by an improved score at least about 4 weeks, at least about 5 weeks, at least about 6 weeks, at least about 7 weeks, or at least about 8 weeks from the initial administration of a compound of formula (I). In certain embodiments, the effects is determined about 4 weeks, about 5 weeks, about 6 weeks, about 7 weeks, about 8 weeks, about 9 weeks, about 10 weeks, about 11 weeks, or about 12 weeks from the initial administration of a compound of formula (I). In certain embodiments, the effects is determined about 6 weeks from the initial administration of a compound of formula (I).

In certain embodiments, the p-value of the improvement is between 0.0001 and 0.05 or less than or equal to 0.05.

In some embodiments the improvement in NPI-NH is determined at about 3-9 weeks, or about 4 weeks, about 5 weeks, about 6 weeks, about 7 or about 8 weeks, of daily administration of a compound of Formula (I).

The methods and uses of the present invention are preferably intended for use in humans. However, it will be understood that the methods may also be applied to non-human animals, including non-human primates, and companion animals including dogs, cats, horses and the like. The recipients of the compounds and compositions are referred herein with the interchangeable terms “patient”, “recipient”, “individual”, and “subject”. These four terms are used interchangeably and refer to any human or animal (unless indicated otherwise), as defined herein.

### Compounds

The methods of the invention comprise administering a compound of Formula (I)

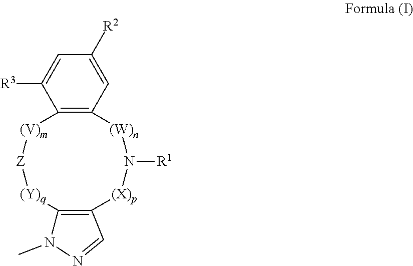

wherein:

V is NH, CH₂ or a direct bond;

W is NH, CH₂ or a direct bond;

X is NH, CH₂ or a direct bond;

Y is NH, CH₂ or a direct bond;

Z is selected from: NH, O, S, S(O), SO₂ or a direct bond;

R¹ is selected from H or C(O)R⁴;

R² is selected from: H, OH, halogen, an optionally substituted C₁₋₅ alkyl or an optionally substituted OC₁₋₅alkyl;

R³ is selected from: H, OH, halogen, an optionally substituted C₁₋₅ alkyl or an optionally substituted OC₁₋₅alkyl;

R⁴ is an optionally substituted C₁₋₅ alkyl;

m is 0 or 1;

n is 0 or 1;

p is 0 or 1; and

q is 0 or 1.

In any embodiment, compounds of formula (I) may be provided as compounds of Formula (Ia):

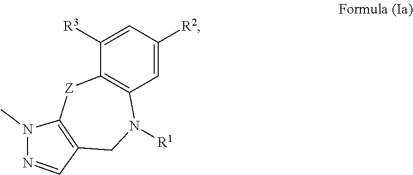

wherein:

Z is selected from: NH, O, S, S(O) or SO₂;

R¹ is selected from H or C(O)R⁴;

R² is selected from: H, a halogen, an optionally substituted C₁₋₅ alkyl, or an optionally substituted OC₁₋₅alkyl;

R³ is selected from: H, a halogen, an optionally substituted C₁₋₅ alkyl, or an optionally substituted OC₁₋₅ alkyl; and

R⁴ is an optionally substituted C₁₋₅ alkyl, or a salt, tautomer, N-oxide, solvate, polymorph and/or prodrug thereof.

In any embodiment, compounds of formula (I) may be provided as compounds of Formula (Ib):

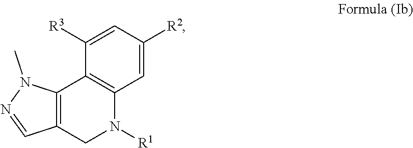

wherein:

R¹ is selected from H or C(O)R⁴;

R² is selected from: H, OH, halogen, an optionally substituted C₁₋₅ alkyl or an optionally substituted OC₁₋₅ alkyl;

R³ is selected from: H, OH, halogen, an optionally substituted C₁₋₅ alkyl or an optionally substituted OC₁₋₅ alkyl; and

R⁴ is an optionally substituted C₁₋₅alkyl, or a salt, tautomer, N-oxide, solvate, polymorph and/or prodrug thereof.

In any embodiment, compounds of formula (I) may be provided as compounds of Formula (Ic):

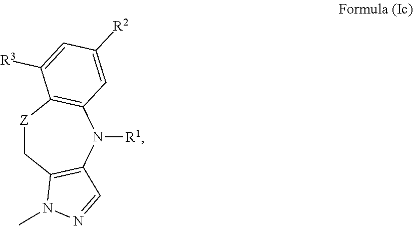

wherein:

Z is selected from: NH, O, S, S(O) or SO₂;

R¹ is selected from H or C(O)R⁴;

R² is selected from: H, OH, halogen, an optionally substituted C₁₋₅ alkyl or an optionally substituted OC₁₋₅alkyl;

R³ is selected from: H, OH, halogen, an optionally substituted C₁₋₅ alkyl or an optionally substituted OC₁₋₅ alkyl; and

R⁴ is an optionally substituted C₁₋₅ alkyl, or a salt, tautomer, N-oxide, solvate, polymorph and/or prodrug thereof.

In any embodiment, compounds of formula (I) may be provided as compounds of Formula (Id):

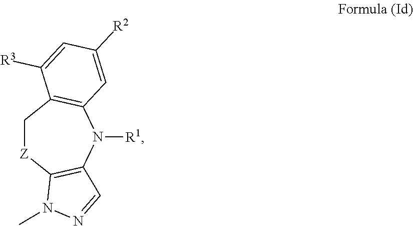

wherein:

Z is selected from: NH, O, S, S(O) or SO₂;

R¹ is selected from H or C(O)R⁴;

R² is selected from: H, OH, halogen, an optionally substituted C₁₋₅ alkyl or an optionally substituted OC₁₋₅alkyl;

R³ is selected from: H, OH, halogen, an optionally substituted C₁₋₅ alkyl or an optionally substituted OC₁₋₅ alkyl; and

R⁴ is an optionally substituted C₁₋₅ alkyl, or a salt, tautomer, N-oxide, solvate, polymorph and/or prodrug thereof.

In any embodiment, compounds of formula (I) may be provided as compounds of Formula (Ie):

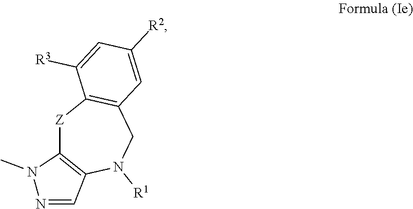

wherein:

Z is selected from: NH, O, S, S(O) or SO₂;

R¹ is selected from H or C(O)R⁴;

R² is selected from: H, OH, halogen, an optionally substituted C₁₋₅ alkyl or an optionally substituted OC₁₋₅alkyl;

R³ is selected from: H, OH, halogen, an optionally substituted C₁₋₅ alkyl or an optionally substituted OC₁₋₅ alkyl; and

R⁴ is an optionally substituted C₁₋₅ alkyl, or a salt, tautomer, N-oxide, solvate, polymorph and/or prodrug thereof.

In some embodiments, compounds of formula (I) may be provided as compounds of Formula (If)

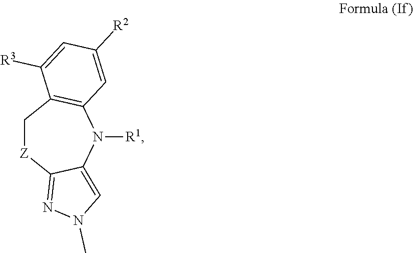

wherein:

Z is selected from: NH, O, S, S(O) or SO₂;

R¹ is selected from H or C(O)R⁴;

R² is selected from: H, OH, halogen, an optionally substituted C₁₋₅ alkyl or an optionally substituted OC₁₋₅ alkyl;

R³ is selected from: H, OH, halogen, an optionally substituted C₁₋₅ alkyl or an optionally substituted OC₁₋₅ alkyl; and

R⁴ is an optionally substituted C₁₋₅alkyl.

In some embodiments the compound of Formula (I) is a compound of Formula (Ia), or a salt or prodrug thereof.

In some embodiments the compound of Formula (I) is a compound of Formula (Ib), or a salt or prodrug thereof.

In some embodiments the compound of Formula (I) is a compound of Formula (Ic), or a salt or prodrug thereof.

In some embodiments the compound of Formula (I) is a compound of Formula (Id), or a salt or prodrug thereof.

In some embodiments the compound of Formula (I) is a compound of Formula (Ie), or a salt or prodrug thereof.

In some embodiments the compound of Formula (I) is a compound of Formula (If), or a salt or prodrug thereof.

In some embodiments V is NH.

In some embodiments V is CH₂

In some embodiments V is a direct bond;

In some embodiments W is NH.

In some embodiments W is CH₂.

In some embodiments W is a direct bond.

In some embodiments X is NH.

In some embodiments X is CH₂.

In some embodiments X is a direct bond

In some embodiments Y is NH.

In some embodiments Y is CH₂.

In some embodiments Y is a direct bond

In some embodiments, Z is NH.

In some embodiments, Z is O.

In some embodiments, Z is S.

In some embodiments, Z is S(O).

In some embodiments, Z is SO₂.

In some embodiments, R¹ is hydrogen.

In another embodiment, R¹ is C(O)R⁴.

In some embodiments, R¹ is C(O)R⁴ and R⁴ is an optionally substituted C₁₋₅ alkyl selected from any one of: methyl, ethyl, propyl, isopropyl, n-butyl, sec-butyl, tent-butyl, n-pentyl, neopentyl, iso-pentyl and tent-pentyl.

In some embodiments, R¹ is C(O)R⁴ and R⁴ is an optionally substituted methyl.

In some embodiments, R² is hydrogen.

In some embodiments, R² is a hydroxyl group.

In some embodiments, R² is a halogen.

In some embodiments, R² is fluorine.

In some embodiments, R² is chlorine.

In some embodiments, R² is an optionally substituted C₁₋₅alkyl.

In some embodiments, R² is an optionally substituted methyl.

In another embodiment, R² is an optionally substituted OC₁₋₅alkyl.

In another embodiment, R² is an optionally substituted methoxy group.

In one embodiment, R³ is hydrogen.

In some embodiments, R³ is a hydroxyl group.

In some embodiments, R³ is a halogen. For example, in some embodiments, R³ is fluorine. In another embodiment, R³ is chlorine.

In some embodiments, R³ is an optionally substituted C₁₋₅ alkyl. For example, R³ may be an optionally substituted C₁₋₅ alkyl selected from: methyl, ethyl, propyl, isopropyl, n-butyl, sec-butyl, tent-butyl, n-pentyl, neopentyl, iso-pentyl and tent-pentyl. In some embodiments R³ is be an optionally substituted methyl.

In some embodiments, R³ is an optionally substituted OC₁₋₅ alkyl. For example, R³ may be an optionally substituted OC₁₋₅alkyl selected from: methoxy, ethoxy, propyloxy, isopropyloxy, n-butyloxy, sec-butyloxy, tert-butyloxy, n-pentyloxy, neopentyloxy, iso-pentyloxy and tent-pentyloxy groups. In some embodiments R³ is an optionally substituted methoxy group.

In some embodiments the compound of Formula (I) is selected from:

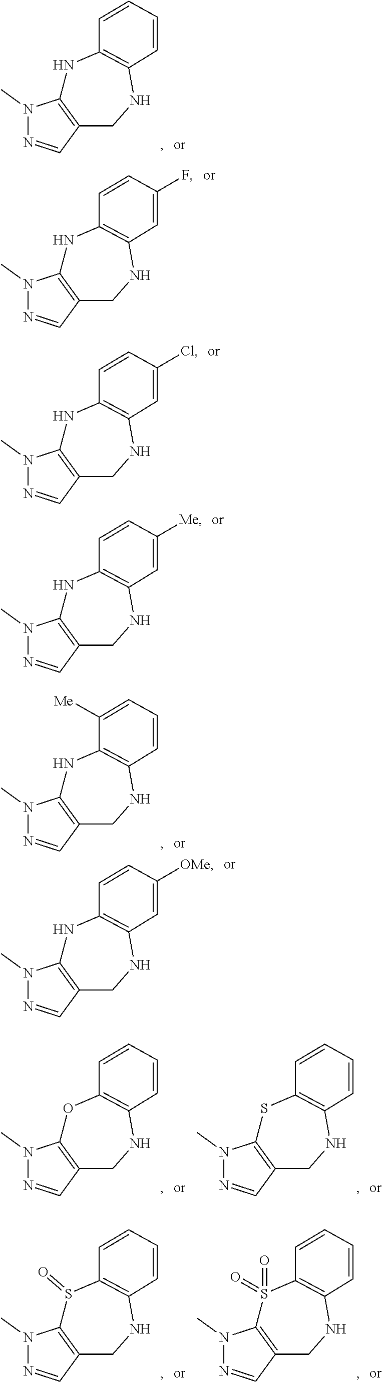

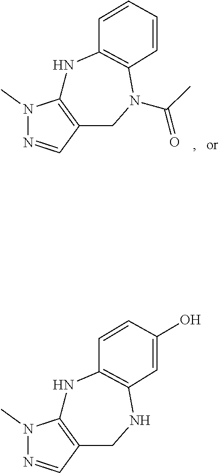

or a salt or prodrug thereof.

In another embodiment, the compound of Formula (I) is selected from:

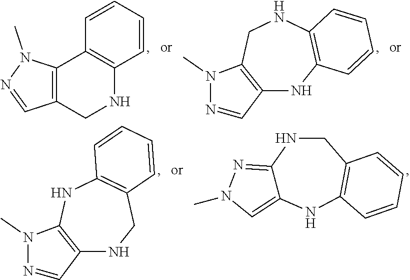

or a salt or prodrug thereof.

In another embodiment compound of Formula (I) is selected from:

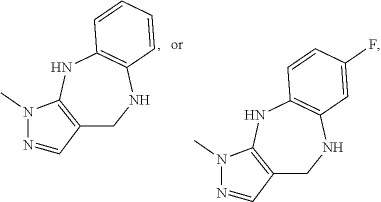 [SMILES: Cn1ncc2c1Nc1ccccc1NC2 ; Cn1ncc2c1Nc1ccc(F)cc1NC2]

or a salt or prodrug thereof. For example, in some embodiments the compound of Formula (I) is:

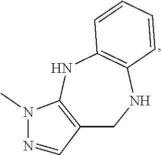

or a salt or prodrug thereof.

The methods may comprise administering the compound of Formula (I) in any pharmaceutically acceptable form. In some embodiments, the compound of Formula (I) is provided in the form of a pharmaceutically acceptable salt, solvate, N-oxide, polymorph, tautomer or prodrug thereof, or a combination of these forms in any ratio.

In some embodiments the compound of Formula (I) is a salt, for example a pharmaceutically acceptable salt.

Suitable pharmaceutically acceptable salts include, but are not limited to: salts of pharmaceutically acceptable inorganic acids such as: hydrochloric, sulfuric, phosphoric, nitric, carbonic, boric, sulfamic, and hydrobromic acids; or salts of pharmaceutically acceptable organic acids such as acetic, propionic, butyric, tartaric, maleic, hydroxymaleic, fumaric, isethionic, malic, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids.

Base salts include, but are not limited to: those formed with pharmaceutically acceptable cations, such as: sodium, potassium, lithium, calcium, magnesium, zinc, ammonium and alkylammonium; salts formed from triethylamine; alkoxyammonium salts such as those formed with ethanolamine; and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1^{st} edition, 2002, Wiley-VCH.

Basic nitrogen-containing groups in Formula (I) (or basic nitrogen-containing groups in a compound of Formula (Ia), Formula (Ib), Formula (Ic), Formula (Id), Formula (Ie), or Formula (If)), may be quarternised with such agents as C₁₋₆alkyl halide, such as methyl, ethyl, propyl, and butyl chlorides, bromides and iodides; dialkyl sulfates like dimethyl and diethyl sulfate; and others known in the art.

In some embodiments the compound of Formula (I) is a salt of a compound selected from:

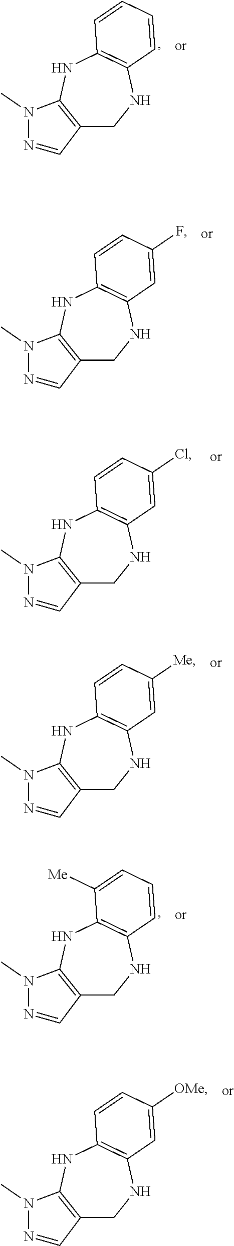 [SMILES: Cn1ncc2c1Nc1ccccc1NC2 ; Cn1ncc2c1Nc1ccc(F)cc1NC2 ; Cn1ncc2c1Nc1ccc(Cl)cc1NC2 ; Cc1ccc2c(c1)NCc1cnn(C)c1N2 ; Cc1cccc2c1Nc1c(cnn1C)CN2 ; COc1ccc2c(c1)NCc1cnn(C)c1N2]

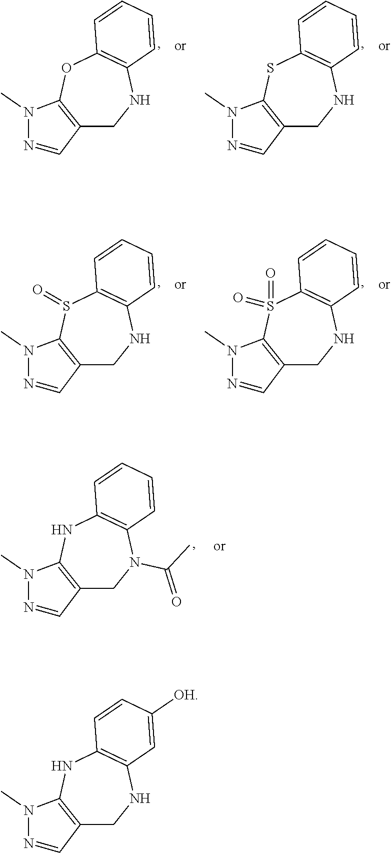 [SMILES: Cn1ncc2c1Oc1ccccc1NC2 ; Cn1ncc2c1Sc1ccccc1NC2 ; Cn1ncc2c1S(=O)c1ccccc1NC2 ; Cn1ncc2c1S(=O)(=O)c1ccccc1NC2 ; CC(=O)N1Cc2cnn(C)c2Nc2ccccc21 ; Cn1ncc2c1Nc1ccc(O)cc1NC2]

In another embodiment, the compound of Formula (I) is a salt of a compound selected from:

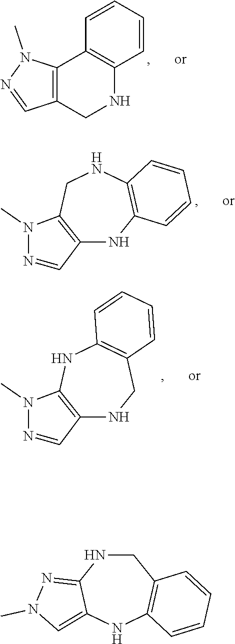

In another embodiment, the compound of Formula (I) is a salt of a compound selected from:

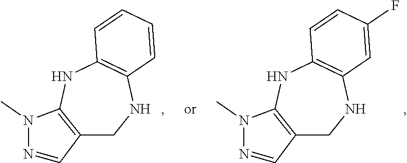 [SMILES: Cn1ncc2c1Nc1ccccc1NC2 ; Cn1ncc2c1Nc1ccc(F)cc1NC2]

For example, in some embodiments, the salt of a compound of Formula (I) is a salt of

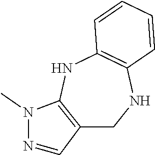

Whilst, in yet another embodiment, the salt of a compound of Formula (I) is a salt of

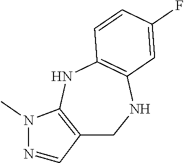 [SMILES: Cn1ncc2c1Nc1ccc(F)cc1NC2]

In some embodiments the compound of Formula (I) is a hydrochloride salt.

In some embodiments, the hydrochloride salt is:

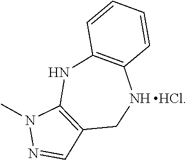

In some embodiments the compound of Formula (I) is a phosphoric acid addition salt.

Prodrugs include compounds wherein an amino acid residue, or a polypeptide chain of two or more (e.g., two, three or four) amino acid residues which are covalently joined to free amino, hydroxy and carboxylic acid groups of compounds of Formula (I). The amino acid residues include the 20 naturally occurring amino acids commonly designated by three letter symbols and also include: 4-hydroxyproline, hydroxylysine, demosine, isodemosine, 3-methylhistidine, norvaline, beta-alanine, gamma-aminobutyric acid, citrulline, homocysteine, homoserine, ornithine and methionine sulfone. Prodrugs also include compounds wherein carbonates, carbamates, amides and alkyl esters, which may be covalently bonded to the above substituents of Formula (I) through the carbonyl carbon prodrug side chain. Prodrugs also include phosphate derivatives of compounds of Formula (I) (such as acids, salts of acids, or esters) joined through a phosphorus-oxygen bond to a free hydroxyl of compounds of Formula (I). It will be understood that the invention disclosed and defined in this specification extends to all alternative combinations of two or more of the individual features mentioned or evident from the text or drawings. All of these different combinations constitute various alternative aspects of the invention.

The compounds of Formula (I) or salts, tautomers, N-oxides, polymorphs or prodrugs thereof may be provided in the form of solvates. Solvates contain either stoichiometric or non-stoichiometric amounts of a solvent, and may be formed during the process of crystallization with pharmaceutically acceptable solvents such as water, alcohols such as methanol, ethanol or isopropyl alcohol, DMSO, acetonitrile, dimethyl formamide (DMF) and the like with the solvate forming part of the crystal lattice by either non-covalent binding or by occupying a hole in the crystal lattice. Hydrates are formed when the solvent is water, alcoholates are formed when the solvent is alcohol. Solvates of the compounds of the present invention can be conveniently prepared or formed during the processes described herein. In general, the solvated forms are considered equivalent to the unsolvated forms for the purposes of the compounds and methods provided herein.

The compound of Formula (I) or salts, tautomers, N-oxides, solvates and/or prodrugs thereof that form crystalline solids may demonstrate polymorphism. All polymorphic forms of the compounds, salts, tautomers, N-oxides, solvates and/or prodrugs may be used in the methods of the invention.

The compound of Formula (I) may demonstrate tautomerism. Tautomers are two interchangeable forms of a molecule that typically exist within an equilibrium. Any tautomers of the compounds of Formula (I) may be used in the methods of the invention.

Methods for making compounds of Formula (I) are described in PCT/AU2016/050588, incorporated herein by reference.

As used herein, the term “C₁₋₅ alkyl” either used alone or in compound terms, refers to monovalent straight chain or branched hydrocarbon groups, having 1 to 5 carbon atoms. As understood by a person skilled in the art, the term “C₁₋₅ alkyl” means an alkyl chain with 1, 2, 3, 4 or 5 carbon atoms or a range including any of two of those integers including 1-2, 1-3, 1-4, 1-5, 2-3, 2-4, 2-5, 3-4, 3-5 and 4-5. Suitable alkyl groups include, but are not limited to: methyl, ethyl, propyl, isopropyl, n-butyl, sec-butyl, tent-butyl, n-pentyl, neopentyl, iso-pentyl and tent-pentyl. The C₁₋₄alkyl may be optionally substituted with one or more substituents. The substituents may be in any position of the carbon chain. Suitable substituents include, but are not limited to: OH, NH₂, halogen, NH(C₁₋₅alkyl), N(C₁₋₅ alkyl)₂, CN, NO₂, CO₂H, or OC₁₋₅alkyl.

The terms “hydroxy” and “hydroxyl” refer to the group —OH.

As used herein, the term “OC₁₋₅ alkyl” either used alone or in compound terms, refers to alkoxy groups having 1 to 5 carbon atoms. As understood by a person skilled in the art, the term “OC₁₋₅ alkyl” means an alkoxy group with 1, 2, 3, 4 or 5 carbon atoms or a range including any of two of those integers and including 1-2, 1-3, 1-4, 1-5, 2-3, 2-4, 2-5, 3-4, 3-5 and 4-5. Suitable OC₁₋₅alkyl groups include, but are not limited to, methoxy, ethoxy, propyloxy, isopropyloxy, n-butyloxy, sec-butyloxy, tert-butyloxy, n-pentyloxy, neopentyloxy, iso-pentyloxy and tent-pentyloxy. The C₁₋₅alkyl may be optionally substituted with one or more substituents. The substituents may be in any position of the carbon chain. Suitable substituents include, but are not limited to: OH, NH₂, halogen, NH(C₁₋₅alkyl), N(C₁₋₅ alkyl)₂, CN, NO₂, CO₂H, or OC₁₋₅alkyl.

As used herein, the term “halo” or “halogen” refers to fluorine (fluoro), chlorine (chloro), bromine (bromo) or iodine (iodo).

### Administration

The compounds of Formula (I), or a pharmaceutically acceptable salt, tautomer, N-oxide, solvate, polymorph and/or prodrug thereof, as defined herein, may be administered by any suitable means, for example, orally, rectally, nasally, vaginally, topically (including buccal and sub-lingual), parenterally, such as by subcutaneous, intraperitoneal, intravenous, intramuscular, or intracisternal injection, inhalation, insufflation, infusion or implantation techniques (e.g., as sterile injectable aqueous or non-aqueous solutions or suspensions).

The compounds of the invention may be provided as pharmaceutical compositions including those for oral, rectal, nasal, topical (including buccal and sub-lingual), parenteral administration (including intramuscular, intraperitoneal, sub-cutaneous and intravenous), or in a form suitable for administration by inhalation or insufflation. The compounds of Formula (I), or a pharmaceutically acceptable salt, tautomer, N-oxide, solvate, polymorph and/or prodrug thereof, together with a conventional adjuvant, carrier or diluent, may thus be placed into the form of pharmaceutical compositions and unit dosages thereof, and in such form may be employed as solids, such as tablets or filled capsules, or liquids as solutions, suspensions, emulsions, elixirs or capsules filled with the same, all for oral use, or in the form of sterile injectable solutions for parenteral (including subcutaneous) use.

The pharmaceutical compositions for the administration of the compounds of this invention may conveniently be presented in dosage unit form and may be prepared by any of the methods well known in the art of pharmacy (See, for example, Remington: The Science and Practice of Pharmacy, 21st Ed., 2005, Lippincott Williams & Wilkins). All methods include the step of bringing the active ingredient, for example a compound defined by Formula (I), or a pharmaceutically acceptable salt, tautomer, N-oxide, solvate, polymorph and/or prodrug thereof, into association with the carrier which constitutes one or more accessory ingredients. In general, the pharmaceutical compositions are prepared by uniformly and intimately bringing the active ingredient, for example a compound defined by Formula (I), or a pharmaceutically acceptable salt, tautomer, N-oxide, solvate, polymorph and/or prodrug thereof, into association with a liquid carrier or a finely divided solid carrier or both, and then, if necessary, shaping the product into the desired formulation. In the pharmaceutical composition the active object compound is included in an amount sufficient to produce the desired effect.

Compounds of Formula (I), or a pharmaceutically acceptable salt, tautomer, N-oxide, solvate, polymorph and/or prodrug thereof, may be administered in doses of about 0.001 mg/kg, about 0.005 mg/kg, about 0.01 mg/kg, about 0.05 mg/kg, about 0.1 mg/kg, about 0.15 mg/kg, about 0.2 mg/kg, about 0.5 mg/kg, about 1 mg/kg, about 2 mg/kg, about 3 mg/kg, about 5 mg/kg, about 10 mg/kg, about 15 mg/kg, about 20 mg/kg, about 25 mg/kg or about 30 mg/kg or more, of the body weight of the subject. In some embodiments, the dose may be from any of these amounts to any other amount, such as from about 0.001 mg/kg to about 30 mg/kg, about 0.2 mg/kg to about 30 mg/kg or about 0.2 mg/kg to about 10 mg/kg. It will be understood, however, that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Compounds of Formula (I), or a pharmaceutically acceptable salt, tautomer, N-oxide, solvate, polymorph and/or prodrug thereof, may be provided in an “effective amount”, for example when an appropriate compound is added to a pharmaceutical composition. “Effective amount” is taken to mean an amount of a compound that will elicit a desired biological or medical response of a tissue, system, animal or human that is being sought by the researcher, veterinarian, medical doctor or other clinician administering the compound of a composition including the compound. In some embodiments, the effective amount may be a “therapeutically effective amount” wherein the amount of the object active compound is effective to treat the condition and/or symptom thereof that has manifested in the subject. In other embodiments, the effective amount may be a “prophylactically effective amount” wherein the amount of the object active compound is sufficient to prophylactically treat and/or prevent the onset of the condition and/or a symptom thereof or, if a symptom emerges, cause the severity of the condition and/or symptom thereof to be at a reduced level compared to the average severity of the condition and/or symptom thereof in a population of subjects not having received treatment with the compound of Formula (I) and/or a pharmaceutically acceptable salt, tautomer, N-oxide, solvate, polymorph and/or prodrug thereof.

An “effective amount” is that amount of a compound of Formula (I), or a pharmaceutically acceptable salt, tautomer, N-oxide, solvate, polymorph and/or prodrug thereof, provided herein, the administration of which to a subject, either in a single dose or as part of a series, is effective for treating or preventing aggression, agitation, irritability as described herein. An amount is effective, for example, when its administration results in an amelioration or improvement in the state of agitation or aggression, or any other parameter as described herein.

The “effective amount” will be dependent on a number of factors, including the efficacy of the particular compound, physical condition of the subject to be treated, the formulation of the compound, and/or a professional assessment of the medical situation. The subject's weight and age may also be a factor for the person skilled in the art when determining the amount of compound that the subject should receive.

The phrases “administration of” and or “administering a” compound should be understood to mean providing a compound of Formula (I) (or a compound of Formula (Ia), Formula (Ib), Formula (Ic), Formula (Id), Formula (Ie)), or Formula (If), or a pharmaceutically acceptable salt, tautomer, N-oxide, solvate, polymorph and/or prodrug thereof, to a subject in need thereof.

In any method or use described herein, the compound of Formula (I) (or a compound of Formula (Ia), Formula (Ib), Formula (Ic), Formula (Id), Formula (Ie)), or Formula (If), or a pharmaceutically acceptable salt, tautomer, N-oxide, solvate, polymorph and/or prodrug thereof, may be administered in combination with one or more other pharmaceutical compositions for the treatment or prevention of agitation, aggression, irritability and/or anger.

Examples of suitable drugs for administration in combination with the compound of Formula (I) (or a compound of Formula (Ia), Formula (Ib), Formula (Ic), Formula (Id), Formula (Ie)), or Formula (If), or a pharmaceutically acceptable salt, tautomer, N-oxide, solvate, polymorph and/or prodrug thereof, include, but are not limited to other drugs for use in the management of aggression, agitation, irritability and/or anger in a subject. In certain embodiments, the drug may be an atypical psychotic. Examples of suitable atypical psychotics include olanzapine, aripiprazole, risperidone and ziprasidone.

The compound of Formula (I) (or a compound of Formula (Ia), Formula (Ib), Formula (Ic), Formula (Id), Formula (Ie)), or Formula (If), or a pharmaceutically acceptable salt, tautomer, N-oxide, solvate, polymorph and/or prodrug thereof, may also be used in combination with any other medicament for the treatment of agitation, aggression, irritability and/or anger associated with or caused by a neurodegenerative condition, an acquired brain injury, a chemical injury, or injury resulting from an infection. Where the agitation, aggression, irritability and/or anger is caused by

Alzheimer's disease, the combination may include a cholinersterase inhibitor (e.g., Aricept, Exelon, Razadyne) or memantine (Namenda).

### EXAMPLES

### Example 1: Murine Model of Hyper-Aggressive Behaviour: Methods

### Objectives

To use a murine model of induced hyper-aggressive behaviour to:
(1) test the effect of KNX100 on aggression across a range of doses;
(2) test the effects of orally administered KNX100 on aggression; and
(3) examine the efficacy of KNX100 in selectively reducing aggression compared to risperidone (FDA approved for the treatment of irritability and aggression associated with autism spectrum disorder, regularly used off-label for the treatment of aggression and agitation in the context of Alzheimer's disease).

This model has been chosen as it is considered to have excellent predictive validity toward human aggression and face validity for modelling offensive impulsive aggression in human aggression and it leads specifically to alterations in aggressive behaviour without confounding changes in other behaviours. This model has also been used for the screening/identification of potential serenics for use in treating aggression/agitation in dementias and related disorders.

The isolation-induced aggression model has also been widely used to understand the neurobiology of aggression, which has identified common pathways driving aggression in these mice with those dysregulated in dementia. To provide just one key example, there is considerable evidence demonstrating that dysregulation of 5-HT pathways is heavily involved in isolation induced aggression in male mice, aggression in Alzheimer's mouse models, and aggression in Alzheimer's patients

A dihydrochloride salt of the compound of the following formula:

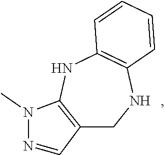

(KNX100) was utilised in the following example and is referred to herein as KNX100 2HCL or KNX100, interchangeably.

### Subjects

Eighty male Swiss mice (ARC, Perth, Australia) aged 6-7 weeks upon arrival were used for objectives (1) and (3) in this study, 30 mice of the same age and strain were used for objective (2). Subjects were housed four per cage in a temperature-controlled colony room (22° C.±1° C.) under standard laboratory conditions (12-hour light/dark cycle; light phase 0700-1900 hours) upon arrival. Mice were housed in standard IVC cages. Mice had ad libitum access to food and water in their home cages. All experiments were performed during the light phase. All experimental procedures were approved by University of Sydney Animal Ethics Committee.

### Induced Aggression Paradigm

After allowing 1 week for acclimatisation, n=64 mice were rendered hyper-aggressive by moving them into single housing, whereas n=16 animals were housed in groups of four. During this time, all mice were weighed at least once per week. One week prior to the commencement of testing mice were given habituation injections with saline (0.9%, 10 mL/kg) to reduce the potential confounding stress and anxiety from injections during testing.

For successful induction of isolation-induced aggression, it was determined that the airflow from the IVC cages must be passed through the filters and exhausted back into the room, which allows exposure of mice to pheromones from mice in other cages. It is believed that this is due to the important role pheromones play in aggression in mice.

### Two Mouse Social Interaction Test and Drug Administration

Testing commenced from 6 weeks post-isolation. All experiments were conducted in testing arenas constructed from acrylic, with matte blue internal walls and floor (400 mm×400 mm×400 mm). The lighting at the centre of the arenas during testing was <10 lux. Prior to each test, one mouse in each pair was marked on its back with a non-toxic green marker, this allowed the automated behavioural tracking software to distinguish between the two mice (see below). On test days, weight and condition matched pairs of unfamiliar mice were administered their drug treatment(s) as described below and were then placed into the testing arena together for a 10 min test session (Examples 2 and 4) or a 5 minute test session (Example 3).

Drugs were administered either via intraperitoneal (i.p.) injection (injection volume 10 ml/kg) or administered orally (as indicated). The testing arenas were cleaned between sessions with ethanol (80%) and F10 disinfectant to eliminate residual odours.

New weight-matched pairs were formed in each experiment with weight differences between mice in each pair being no greater than 5 g. Drug treatments were randomly allocated to pairs and mice were given a one-week washout between tests.

### Drugs

KNX100 2HCL (Wuxi Apptech, Hong Kong, China) was dissolved in saline (0.9%). Risperidone (AK Scientific, CA, USA) was made up into a 1 mg/ml stock solution (10% 0.1 M acetic acid, 90% 0.9% saline and a small amount of NaOH to bring pH up to 6).

### KNX100 Dose Response

A 5 group between-subjects design was utilised. 15 min prior to testing, group-housed mice received vehicle treatment (VEHg) and single housed mice received one of four different doses of KNX100 (0 aka VEHs, 2.5, 5 or 10 mg/kg). The sample size in each group was as follows: group housed (n=12); singled housed administered KNX100 dose 0 (n=14), 0.25 (n=16), 0.5 (n=18), and 10 (n=16) mg/kg, for i.p. administration.

### KNX100 Oral Dosing

For oral dosing, mice were similarly grouped, and 20 mg/kg KNX100 in saline or vehicle (saline) were administered by oral gavage with n=10 mice per group.

### KNX100 vs Risperidone

A 4 group between-subjects design was utilised. Prior to testing, group housed mice received vehicle treatment (VEHg) and single housed mice received vehicle (VEHs), 0.1 mg/kg risperidone, or 10 mg/kg KNX100. Risperidone was administered 30 min prior to testing and vehicle and KNX100 15 min prior to testing. The sample size in each group was as follows: vehicle group housed (n=6); vehicle singled housed (n=12); 0.1 mg/kg risperidone (n=10); and 10 mg/kg KNX100 (n=10).

### Dependent Variables

The behaviours of interest were: (1) time (in seconds) spent fighting; (2) time (in seconds) spent huddling (defined as the mice being in close physical contact with each other while stationary); (3) time (in seconds) spent grooming themselves; and (4) distance travelled (in mm) by each mouse during the experimental session.

### Data Acquisition and Analysis

Experimental sessions were recorded with an overhead camera using CaptureStar (Version 1.00, CleverSys, Virginia, USA). The behaviours detailed above were automatically quantified from the videos using the behavioural tracking software TopScan, SocialScan and AggressionScan (CleverSys, Virginia, USA). Data were analysed using SPSS (Version 24.0, IBM, USA). Data were analysed with a one-way ANOVA and planned contrasts. Significance was set at p≤0.05.

### Example 2: KNX100 Dose Dependently Reduces Aggression

Compared to group housed mice, single housed mice spent significantly more time fighting (p=0.021), huddling (p<0.0001), and grooming (p=0.014). There was no significant difference in distance travelled between group housed and single housed mice (p=0.692).

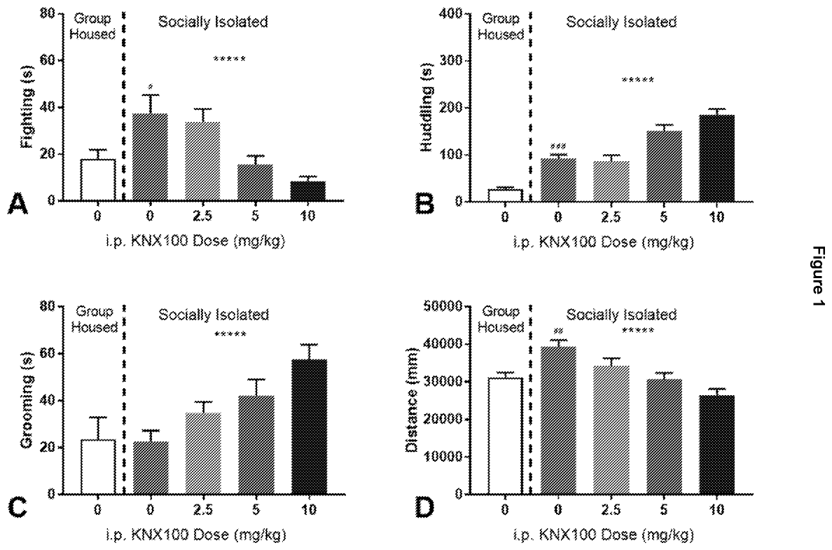

Data from Example 2 are presented in FIG. 1. Single housed mice spent significantly more time fighting than group-housed mice (VEHg vs VEHs, p=0.012). The linear trend contrast was significant (p<0.00001) for fight time, revealing that KNX100 dose dependently inhibited induced aggression in single housed mice.

Single housed mice spent significantly more time huddling than group housed mice (VEHg vs VEHs, p<0.001). The linear trend contrast was significant (p<0.00000001) for huddling, revealing that KNX100 dose dependently increased time spent in this form of close physical contact in single housed mice.

There was no significant difference between single housed and group housed mice in time spent self-grooming (VEHg vs VEHs, p=0.919). The linear trend contrast was significant (p<0.001) for self-grooming, revealing that KNX100 dose dependently increased time spent self-grooming in single housed mice.

Vehicle treated single housed mice travelled significantly farther distance than vehicle treated group housed mice (VEHg vs VEHs, p=0.004). The linear trend contrast was significant (p<0.00001) for distance travelled, revealing that KNX100 dose dependently decreased distance travelled in single housed mice. Note that relative to the group housed mice, none of the doses of KNX100 resulted in a significant difference in distance travelled (vs: 2.5 mg/kg, p=0.262; 5 mg/kg, p=0.858; 10 mg/kg, p=0.092).

### Example 3: Orally Dosed KNX100 Reduces Aggression

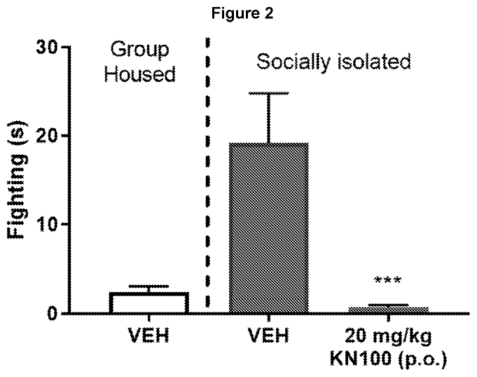

Data from Example 3 are presented in FIG. 2. Vehicle treated single housed mice spent significantly more time fighting than vehicle treated group housed mice (p=0.0011). Mice treated with KNX100 20 mg/kg (p.o.) spent significantly less time fighting than vehicle treated single housed mice (p=0.0004) and did not differ significantly in time spent fighting compared to vehicle treated group housed mice (p=0.725).

### Example 4: KNX100 is as Effective as Risperidone at Reducing Aggression and is Less Sedating

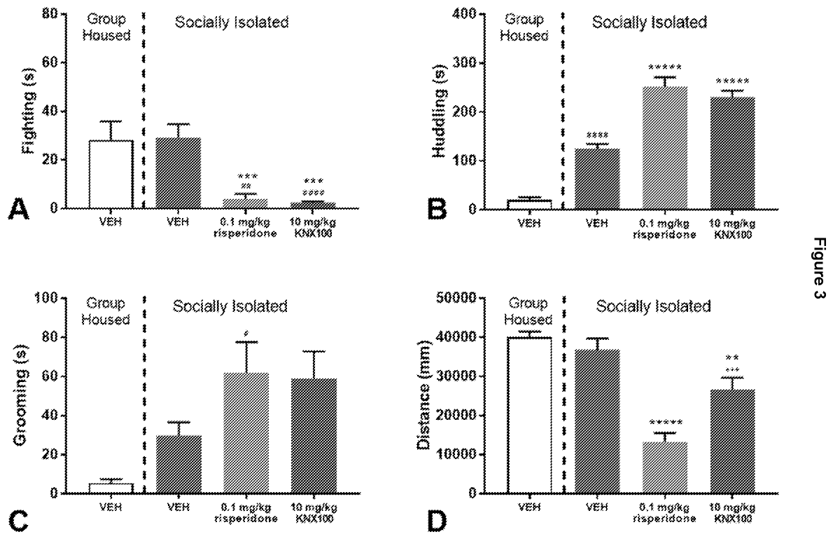

Data from Example 4 are presented in FIG. 3. The vehicle treated single housed mice did not differ in time spent fighting compared to the vehicle treated group housed mice (p=0.896). However, both 0.1 mg/kg risperidone and 10 mg/kg KNX100 groups spent less time fighting relative to vehicle treated single housed mice (p<0.001 and 0.0001, respectively) and vehicle treated group housed mice (p<0.01 and 0.001, respectively). There was no significant difference in time spent fighting between the 0.1 mg/kg risperidone and 10 mg/kg KNX100 groups (p=0.807).

Vehicle treated single housed mice spent significantly more time huddling than vehicle treated group housed mice (p<0.0001). Both the 0.1 mg/kg risperidone and 10 mg/kg KNX100 treated mice spent significantly more time huddling than the vehicle treated single housed mice (p<0.0000001 and 0.00001, respectively). There was no significant difference in time spent huddling between the risperidone and KNX100 groups (p=0.247).

The vehicle treated single housed mice did not differ in time spent grooming compared to the vehicle treated group housed mice (p=0.191). Mice treated with 0.1 mg/kg risperidone spent significantly more time grooming compared to vehicle treated single housed mouse (p=0.047). Mice treated with 10 mg/kg KNX100 did not differ significantly from vehicle treated group housed mice in time spent grooming (p=0.07). There was no significant different in time spent grooming between the risperidone and KNX100 treated mice (p=0.859).

The vehicle treated single housed mice did not differ in distance travelled compared to the vehicle treated group housed mice (p=0.445). Both 0.1 mg/kg risperidone and 10 mg/kg KNX100 treated mice travelled significantly less distance than the vehicle treated single housed mice (p<0.000001 and 0.01, respectively). However, mice treated with 0.1 mg/kg risperidone also travelled significantly less distance than mice treated with 10 mg/kg KNX100 (p=0.001).

KNX100 appears to offer significant advantages over risperidone, causing at least equivalent inhibition of aggression to risperidone with significantly less suppression of locomotor activity.

### Example 5: Evaluation of KNX100 in Alzheimer's Disease Aggression Mouse Model Animals

Male APP/PS1 and WT mice (˜3 months of age) were used to assess the effect of KNX100 on aggression in a mouse model of Alzheimer's Disease.

APP/PS1 are double transgenic mice expressing a chimeric mouse/human amyloid precursor protein (APP) and presenilin (PS1). These mice exhibit increased levels of AR and tau in CSF and brain tissue and associated behavioural abnormalities and are therefore frequently used as a model for studying Alzheimer's Disease.

Male C57BL/6J mice were purchased from Jackson Laboratory at 7-8 weeks of age and were used as the intruders in this study. The APP/PS1 mice and wild-type control mice were single-housed and C57BL/6J mice were group-housed (4 per cage) in a light-controlled environment (12/12 light/dark cycle) with access to food and water ad libitum. Tests were performed during the animal's light cycle.

### Test and Reference Compounds

The reference compound, risperidone (0.05 mg/kg), was dissolved in 25% hydroxypropyl-8-cyclodextrin and injected intraperitoneally (IP), 30 minutes prior to test at a dose volume of 10 ml/kg.

The test compound, KNX100 was provided in its phosphate salt form. Doses of 2.5, 5, and 10 mg/kg freebase equivalent were dissolved in saline and injected IP, at a dose volume of 10 ml/kg, 15 minutes prior to the test.

### Treatment Groups

### Treatment Groups Were as Follows:

WT—saline (IP)
APP/PS1—saline (IP)
APP/PS1—KNX100 (2.5 mg/kg, IP)
APP/PS1—KNX100 (5 mg/kg, IP)
APP/PS1—KNX100 (10 mg/kg, IP)
APP/PS1—Risperidone (0.05 mg/kg, IP)
n=12 in each group.

### Pre-Screen AD Mice in Resident Intruder (RI) Test

All APP/PS1 mice were pre-screened using the Resident-Intruder (RI) paradigm. The pre-screen RI session was performed in the home cage of the resident APP/PS1 mouse. All RI sessions were carried out under red lights.

Briefly, all mice were acclimated to red-light conditions for 60 minutes prior to testing. During the RI test, an unfamiliar intruder C57BL/6J mouse was placed directly into the home cage of the APP/PS1 resident mouse for 300 seconds. During the 5-minute session, the latency time to attack (attack was defined at a bite), and the total number (frequency) of attacks, were recorded manually by an experimenter blinded to the treatment groups. Immediately after testing, mice were separated, and the intruder mouse was placed back into his home cage. All mice that were wounded were treated according to IACUC guidelines.

This pre-screen test was necessary to ensure a uniform distribution of aggressive behaviour across the APP/PS1 treatment groups (data not shown). APP/PS1 mice that showed a latency to attack over 150 seconds were not used for further RI testing

### Effect of Treatment on Aggression in AD Mice

One week after the pre-screen RI session, AD resident mice were administered vehicle, test compound, or risperidone. The RI test session was performed as described above following the appropriate pretreatment time.

### Statistical Analysis

Body weights were recorded at each time point as specified above. For aggression, the latency to attack and the total number of attacks were recorded.

Data were analyzed by analysis of variance (ANOVA) followed by post-hoc comparisons when appropriate. An effect was considered significant if p<0.05. Data are presented as the mean±standard error of the mean (SEM).

### Results

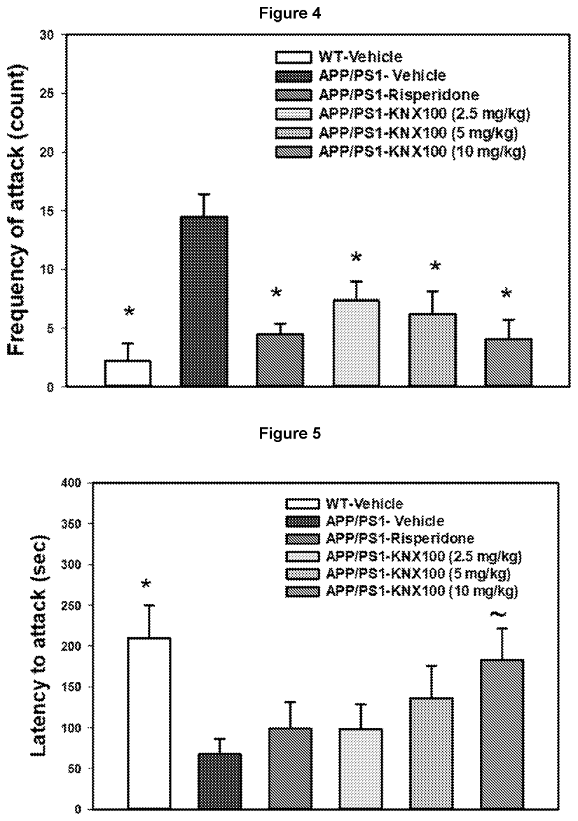

The effects of KNX100 on the number of attacks on the intruder mouse are shown in FIG. 4. A one-way ANOVA across all treatment groups revealed a significant main effect of treatment. Dunnett's post-hoc test revealed that compared to APP/PS1 vehicle mice, WT mice had significantly lower frequency of attacks. Treatment with risperidone or KNX100 (2.5, 5 or 10 mg/kg) decreased the number of attacks in APP/PS1 mice compared to vehicle.

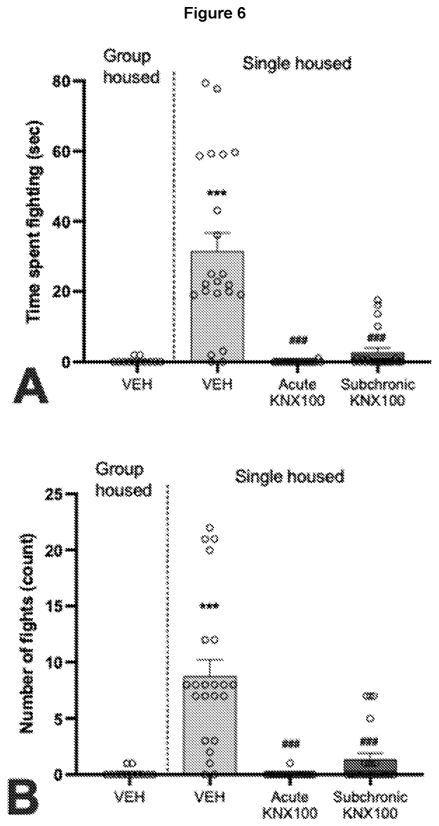

The effects of KNX100 on the latency to attack the intruder mouse are shown in FIG. 5. A one-way ANOVA across all treatment groups found a significant treatment effect. Dunnett's post-hoc test revealed that compared to APP/PS1 vehicle mice, WT mice had longer latency to attack. KNX100 (10 mg/kg) showed a trend to increasing the latency to attack (p=0.086). Please note that if Fisher's LSD post hoc test was used the effect reaches significance (p=0.022).

This study evaluated the effects of acute injection of KNX100 on aggression in APP/PS1 mice. All doses of KNX100 (2.5, 5 and 10 mg/kg) robustly reduced the number of aggressive attacks. The highest dose tested (10 mg/kg) also tended to increase the latency to attack intruders in the RI test.

### Example 6: Subchronic Dosing of KNX100

This study used the same model and testing procedure for the two-mouse social interaction test as described in Example 1, and using a phosphate salt form of KNX100. The purpose of this study was to assess whether KNX100 maintains its efficacy at reducing aggression when dosed repeatedly.

Mice in the VEH (group housed) and VEH (single housed) conditions received 6 days of once daily saline via oral gavage (p.o.) then on the 7th day received a final saline administration p.o. 30 minutes before undergoing a 10-minute two-mouse social interaction test. Mice in the acute KNX100 (single housed) condition received 6 days of once daily saline p.o. then on the 7th day received KNX100 phosphate at a dose of 15 mg/kg (freebase equivalent) p.o. 30 minutes before undergoing a 10-minute two-mouse social interaction test. Mice in the subchronic KNX100 (single housed) condition received 6 days of once daily KNX100 phosphate at a dose of 15 mg/kg (freebase equivalent) p.o. then on the 7th day received KNX100 phosphate at a dose of 15 mg/kg (freebase equivalent) p.o. 30 minutes before undergoing a 10-minute two mouse social interaction test.

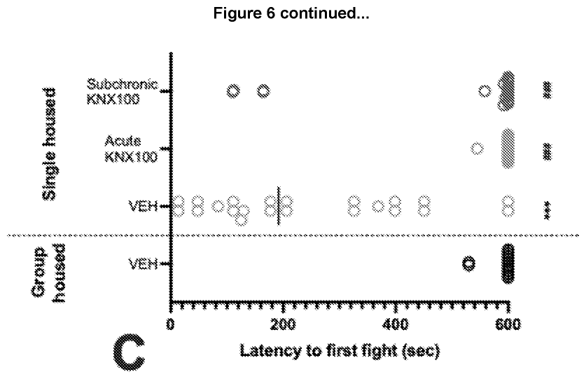

Data are presented in FIG. 6. VEH (single housed) mice spent more time fighting (p<0.001), had more fights (p<0.001) and had a shorter latency to their first fight (p<0.001) than the VEH (group housed) mice. Both acute and subchronic KNX100 prevented the increased time spent fighting (both p<0.001), greater number of fights (both p<0.001) and reduced latency to first fight (both p<0.001) in the single housed mice. There was no significant difference in efficacy between acute and subchronic KNX100 for duration spent fighting (p=0.514), number of fights (p=0.281), or latency to first fight (p=0.055).

These findings demonstrate that KNX100 dosed orally maintains full efficacy at reducing aggressive behaviour after repeated dosing.

### Example 7: Treatment of Aggression in Neurodevelopmental Disorders Associated with a Genetic Cause or Risk Variant

Numerous single gene point mutations are causally linked to neurodevelopmental disorders in clinical populations, including in patients presenting with increased aggression. One example is a point mutation (R451C) in the neuroligin-3 gene which has been identified in clinical populations as having high penetrance for neurodevelopmental disorders.

Mice with the NL3R451C point mutation recapitulate many of the symptoms observed in patients with the mutation, and the mice exhibit heightened aggression. These mice have previously been used as a model for studying abnormal behavioural phenotypes associated with neurodevelopmental disorders and in order to identify suitable clinical candidates.

KNX100 is assessed for its ability to inhibit the increased number of fights, duration spent fighting, and latency to first attack in NL3R451C mice, compared to wild-type controls.

Briefly, six primary experimental conditions of interest are investigated: WT mice treated with vehicle (WT-VEH)
NL3R451C (NL3) mice treated with vehicle (NL3-VEH)
NL3 mice treated positive control risperidone (0.05-0.1 mg/kg i.p.)
NL3 mice treated with 1.845 mg/kg freebase equivalent KNX100 phosphate i.p. or 3.75 mg/kg p.o. (NL3-KNX100-D1)
NL3 mice treated with 3.69 mg/kg freebase equivalent KNX100 phosphate i.p. or 7.5 mg/kg p.o. (NL3-KNX100-D2)
NL3 mice treated with 7.38 mg/kg freebase equivalent KNX100 phosphate i.p. or mg/kg p.o. (NL3-KNX100-D3)

A resident intruder test is then used to assess the effects of treatment on the heightened aggression in the NL3 mice. In addition to the resident WT and NL3 mice, male C57BL/6 mice are used as intruder mice for this test. The resident mice (WT and NL3 mice) are isolated for at least 1 week prior to testing. WT or NL3 mice receive their treatment either 15 min (for i.p.) or 30 min (for p.o.) before testing. An intuder mouse is then placed into the homecage of the resident mouse for a 5-10 min aggression test. The behaviours of the WT or NL3 resident mouse are assessed and include the total time spent fighting, the total number of fights, and the latency to the first fight. NL3 mice treated with KNX100 will show reductions in some or all of these behavioural measures.

In NL3R451C mice, KNX100 is shown to inhibit fighting incidents, duration of fighting, and latency to first attack, similarly to the outcome observed in the AD mouse model and the social isolation mouse model, described elsewhere herein. Thus, KNX100 is expected to be a useful clinical candidate for treatment of aggression associated or caused by neurodevelopmental disorders associated with a genetic cause or risk variant.

Similar experimental protocols are applied in the context of other animal models of neurodevelopmental disorders associated with a genetic cause or risk variant and where the animals display heightened aggression. Some examples are: the UBE3A mouse model of Angelman syndrome (Simchi & Kaphzan et al., (2021) Scientific Reports, 11: 47); the SHANK3 mouse model (Zhang et al., (2021) Translational Psychiatry, 11:99); the Neurexin lalpha mouse model (Armstrong et al., (2020) Genes Brain and Behavior, 19:e12630); the Fragile X Syndrome mouse model (Cogram et al., (2019) Frontiers in Behavioural Neuroscience, 13:141); the Dlgap2 mouse model (Jian-Xie et al., (2014) Molecular Autism, 5:32); and the Oxtr KO mouse model (Sala et al., (2011) Biological Psychiatry, 69:875-82). Dosing and conditions are the same as for the NL3 mouse model described above, and either a resident intruder test or social interaction test in a neutral context will be used to determine the effect of KN100 on aggressive behaviour.

### Example 8:Treatment of Aggression in ADHD

Increased aggression is commonly observed in patients with ADHD. This increased aggression is also observed in several animal models of ADHD. One such model is the neonatal 6-hydroxydopamine (6-OHDA) lesion mouse model.

Briefly, intracerebroventricular infusion of 6-OHDA into brains of mice around postnatal day 5 results in a biological ADHD phenotype (e.g. dopamine depletion) and a behavioural phenotype (e.g. hyperactivity, impaired attention, impulsivity, aggression, and learning and memory deficits) in the majority of mice from postweaning onwards.

Five primary experimental conditions of interest are investigated:
Sham lesion mice treated with vehicle
6-OHDA lesion mice treated with vehicle
6-OHDA lesion mice treated with 1.845 mg/kg freebase equivalent KNX100 phosphate i.p. or 3.75 mg/kg p.o.
6-OHDA lesion mice treated with 3.69 mg/kg freebase equivalent KNX100 phosphate i.p. or 7.5 mg/kg p.o.
6-OHDA lesion mice treated with 7.38 mg/kg freebase equivalent KNX100 phosphate i.p. or 15 mg/kg p.o.

During adolescence, mice undergo a social interaction test to test the effects of KNX100 on increased aggression the ADHD model mice (6-OHDA lesion mice) compared to the control mice (sham lesion mice). Mice receive treatment either 15 min (for i.p.) or 30 min (for p.o.) before the social interaction test. The behaviours of the control or ADHD model mouse are assessed and include the total time spent fighting, the total number of fights, and the latency to the first fight. ADHD model mice treated with KNX100 will show reductions in some or all of these behavioural measures.

In ADHD model mice, KNX100 is shown to inhibit fighting incidents, duration of fighting, and latency to first attack, similarly to the outcome observed in the AD mouse model and the social isolation mouse model, described elsewhere herein. Thus, KNX100 is expected to be a useful clinical candidate for treatment of aggression associated or caused by ADHD.

It will be understood that the invention disclosed and defined in this specification extends to all alternative combinations of two or more of the individual features mentioned or evident from the text or drawings. All of these different combinations constitute various alternative aspects of the invention.

### Clauses

1. A method of treating or preventing aggression, agitation, irritability and/or anger in a subject, comprising administering to a subject in need thereof, a therapeutically effective amount of a compound of Formula (I):

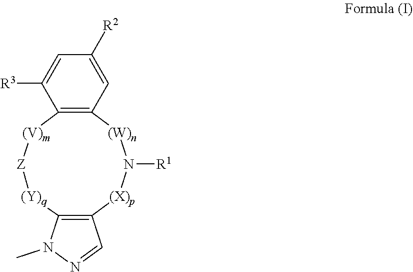

wherein:

V is NH, CH₂ or a direct bond;

W is NH, CH₂ or a direct bond;

X is NH, CH₂ or a direct bond;

Y is NH, CH₂ or a direct bond;

Z is selected from: NH, O, S, S(O), SO₂ or a direct bond;

R¹ is selected from H or C(O)R⁴;

R² is selected from: H, OH, halogen, an optionally substituted C₁₋₅ alkyl or an optionally substituted OC₁₋₅alkyl;

R³ is selected from: H, OH, halogen, an optionally substituted C₁₋₅ alkyl or an optionally substituted OC₁₋₅alkyl;

R⁴ is an optionally substituted C₁₋₅ alkyl;

m is 0 or 1;

n is 0 or 1;

p is 0 or 1; and

q is 0 or 1;

or a pharmaceutically acceptable salt or prodrug thereof, thereby treating the aggression, agitation, irritability and/or anger in the subject.

2. Use of a compound of formula (I)

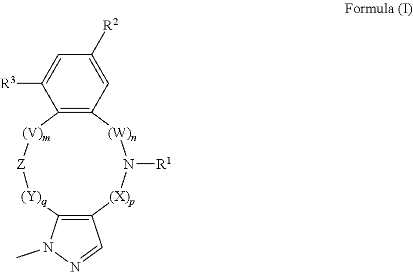

wherein:

V is NH, CH₂ or a direct bond;

W is NH, CH₂ or a direct bond;

X is NH, CH₂ or a direct bond;

Y is NH, CH₂ or a direct bond;

Z is selected from: NH, O, S, S(O), SO₂ or a direct bond;

R¹ is selected from H or CO(O)R⁴;

R² is selected from: H, OH, halogen, an optionally substituted C₁₋₅ alkyl or an optionally substituted OC₁₋₅alkyl;

R³ is selected from: H, OH, halogen, an optionally substituted C₁₋₅ alkyl or an optionally substituted OC₁₋₅alkyl;

R⁴ is an optionally substituted C₁₋₅ alkyl;

m is 0 or 1;

n is 0 or 1;

p is 0 or 1; and

q is 0 or 1;

or a pharmaceutically acceptable salt or prodrug thereof,

in the manufacture of a medicament for treating or preventing aggression, agitation, irritability and/or anger in a subject.

3. A compound of formula (I) or pharmaceutical composition comprising the same,

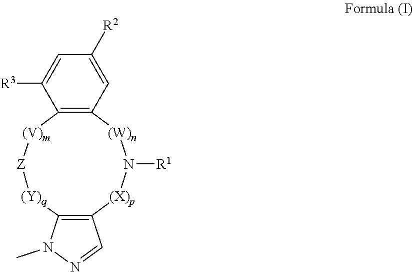

for use in treating or preventing aggression, agitation, irritability and/or anger in a subject,

wherein:

V is NH, CH₂ or a direct bond;

W is NH, CH₂ or a direct bond;

X is NH, CH₂ or a direct bond;

Y is NH, CH₂ or a direct bond;

Z is selected from: NH, O, S, S(O), SO₂ or a direct bond;

R¹ is selected from H or C(O)R⁴;

R² is selected from: H, OH, halogen, an optionally substituted C₁₋₅ alkyl or an optionally substituted OC₁₋₅alkyl;

R³ is selected from: H, OH, halogen, an optionally substituted C₁₋₅ alkyl or an optionally substituted OC₁₋₅alkyl;

R⁴ is an optionally substituted C₁₋₅ alkyl;

m is 0 or 1;

n is 0 or 1;

p is 0 or 1; and

q is 0 or 1;

or a pharmaceutically acceptable salt or prodrug thereof.

4. The method of clause 1, use of clause 2, or the compound or pharmaceutical composition for the use of clause 3, wherein the compound of Formula (I) is a compound of Formula (Ia),

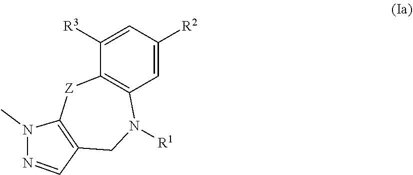

wherein:

Z is selected from: NH, O, S, S(O) or SO₂;

R¹ is selected from H or C(O)R⁴;

R² is selected from: H, OH, halogen, an optionally substituted C₁₋₅ alkyl or an optionally substituted OC₁₋₅ alkyl;

R³ is selected from: H, OH, halogen, an optionally substituted C₁₋₅ alkyl or an optionally substituted OC₁₋₅alkyl; and

R⁴ is an optionally substituted C₁₋₅ alkyl,

or a pharmaceutically acceptable salt or prodrug thereof

5. The method, use, or the compound or pharmaceutical composition for use, of clause 4, wherein the compound of Formula (I) is:

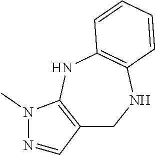

or a pharmaceutically acceptable salt or prodrug thereof.

6. The method, use or compound or pharmaceutical composition for use, of any one of clauses 1 to 5, wherein the aggression, agitation, irritability and/or anger is associated with or caused by cognitive decline, a cognitive disorder and/or an intellectual disability in the subject.

7. The method, use or compound or pharmaceutical composition for use, of clause 6, wherein the cognitive decline, cognitive disorder and/or intellectual disability is associated with or caused by physical change or injury to the brain.

8. The method, use or compound or pharmaceutical composition for use, of clause 6, wherein the cognitive disorder and/or intellectual disability is associated with or caused by a neurodevelopmental disorder, optionally wherein the neurodevelopmental disorder is associated with or caused by one or more genetic mutations or risk variants and/or a physical and/or chemical injury during early development.

9. The method, use or compound or pharmaceutical composition for use, of clause 7, wherein the physical change or injury to the brain is caused by or associated with one or more of: a neurodegenerative condition, an acquired brain injury, a chemical injury, or injury resulting from an infection.

10. The method, use or compound or pharmaceutical composition for use, of clause 9 wherein the chemical injury is associated or caused by alcohol, drugs or neurotoxins.

11. The method, use or compound or pharmaceutical composition for use, of clause 9 wherein the acquired brain injury results from compression or blunt trauma to the brain leading to traumatic brain injury (TBI), ischemic injury resulting from a transient ischemic attack (TIA), ischemic stroke or haemorrhagic stroke.

12. The method, use or compound or pharmaceutical composition for use, of clause 9, wherein the neurodegenerative condition is characterised by the presence of abnormal protein deposits in the brain, including amyloidopathies, synucleinopathies or taupathies.

13. The method, use or compound or pharmaceutical composition for use, of clause 12, wherein the neurodegenerative condition or disorder is selected from the group consisting of: Alzheimer's disease (AD), Lewy-bodies disease (Dementia with Lewy bodies (DLB)), Huntington's disease, Creutzfeldt-Jakob disease (CJD), Gaucher Disease Type 3, and Parkinson's disease.

14. The method, use or compound or pharmaceutical composition for use, of clause 9, wherein the neurodegenerative condition or disorder is selected vascular dementia, frontotemporal dementia or other form of dementia not typically associated with deposition of abnormal protein deposits.

15. The method, use or compound or pharmaceutical composition for use, of clause 9, wherein the aggression, agitation, irritability and/or anger is associated with one or more of AD, vascular dementia, frontotemporal dementia, mixed dementia or Korsakoff syndrome.

16. The method, use or compound or pharmaceutical composition for use, of any one of clauses 1 to 7, wherein the aggression, agitation, irritability and/or anger is associated with AD.

17. The method, use or compound or pharmaceutical composition for use, of clause 8 wherein the neurodevelopmental disorder is selected from the group consisting of: Fragile-X Syndrome, X-Linked Intellectual Disability-Hypotonia-Facial Dysmorphism-Aggressive Behaviour Syndrome, Kleefstra Syndrome, Hunters Syndrome (MPS II), ADNP (activity dependent neuroprotector homeobox) syndrome, Rett syndrome, Autism Spectrum Disorder, Prader-Willi syndrome, Angelman Syndrome, Brunner syndrome, Cri du Chat syndrome, Cornelia de Lange syndrome, Smith-Lemli-Opitz syndrome, Smith-Magenis syndrome, Tuberous Sclerosis Complex, CHARGE syndrome, sotos syndrome, attention-deficit/hyperactivity disorder (ADHD), PTEN associated disorder, cerebral palsy, and fetal alcohol spectrum disorder.

18. The method, use or compound or pharmaceutical composition for use, of any one of clauses 1 to 17 wherein the therapeutically effective amount of the compound of Formula (I), or a pharmaceutically acceptable salt or prodrug thereof, does not simultaneously cause sedation of the subject receiving treatment.

19. The method, use or compound or pharmaceutical composition for use, of any one of clauses 1 to 18, wherein the compound of Formula (I), or a pharmaceutically acceptable salt or prodrug thereof, is administered orally, intranasally, systemically (e.g., subcutaneously, intramuscularly, intraperitoneally, intravenously) or rectally or is for oral, intranasal, systemic or rectal administration.

20. A kit comprising an effective amount of a compound of Formula (I) or a pharmaceutically acceptable salt and/or prodrug thereof, wherein the kit includes written instructions for performing the methods of any one of clauses 1 to 19.

1. A method of treating or preventing aggression, agitation, irritability and/or anger in a subject, comprising administering to a subject in need thereof, a therapeutically effective amount of a compound of Formula (I): 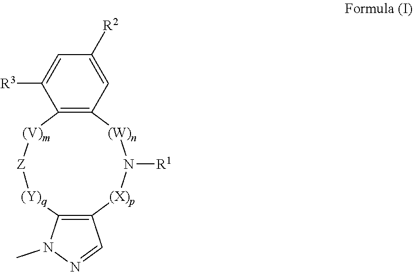
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅alkyl;
R³ is selected from: H, OH, halogen, an optionally substituted C₁₋₅ alkyl or an optionally substituted OC₁₋₅alkyl;
R⁴ is an optionally substituted C₁₋₅ alkyl;
m is 0 or 1;
n is 0 or 1;
p is 0 or 1; and
q is 0 or 1;
or a pharmaceutically acceptable salt or prodrug thereof, thereby treating the aggression, agitation, irritability and/or anger in the subject.

2. Use of a compound of formula (I) 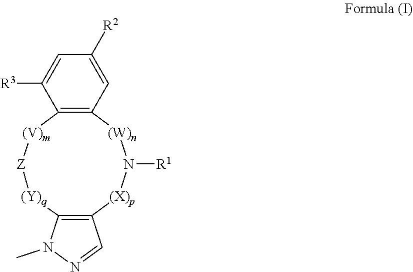
wherein:
V is NH, CH₂ or a direct bond;
W is NH, CH₂ or a direct bond;
X is NH, CH₂ or a direct bond;
Y is NH, CH² or a direct bond;
Z is selected from: NH, O, S, S(O), SO₂ or a direct bond;
R¹ is selected from H or C(O)R⁴;
R² is selected from: H, OH, halogen, an optionally substituted C₁₋₅alkyl or an optionally substituted OC₁₋₅alkyl;
R³ is selected from: H, OH, halogen, an optionally substituted C₁₋₅ alkyl or an optionally substituted OC₁₋₅alkyl;
R⁴ is an optionally substituted C₁₋₅ alkyl;
m is 0 or 1;
n is 0 or 1;
p is 0 or 1; and
q is 0 or 1;
or a pharmaceutically acceptable salt or prodrug thereof,
in the manufacture of a medicament for treating or preventing aggression, agitation, irritability and/or anger in a subject.

3. The method of claim 1, use of claim 2, wherein the compound of Formula (I) is a compound of Formula (Ia), 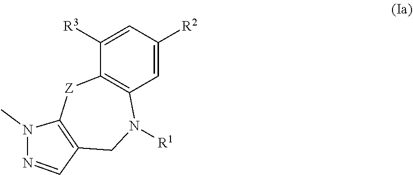
wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl, or a pharmaceutically acceptable salt or prodrug thereof

4. The method or use of claim 3, wherein the compound of Formula (I) is: 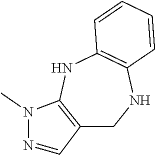
or a pharmaceutically acceptable salt or prodrug thereof.

5. The method or use of any one of claims 1 to 4, wherein the aggression, agitation, irritability and/or anger is associated with or caused by cognitive decline, a cognitive disorder and/or an intellectual disability in the subject.

6. The method or use of claim 5, wherein the cognitive decline, cognitive disorder and/or intellectual disability is associated with or caused by physical change or injury to the brain.

7. The method or use of claim 5, wherein the cognitive decline, cognitive disorder and/or intellectual disability is caused by one or more genetic mutations or risk variants.

8. The method or use of any one of claims 5 to 7, wherein the cognitive disorder and/or intellectual disability is associated with or caused by a neurodevelopmental disorder.

9. The method or use of claim 6, wherein the physical change or injury to the brain is caused by or associated with one or more of: a neurodegenerative condition, an acquired brain injury, a chemical injury, or injury resulting from an infection.

10. The method or use of claim 9, wherein the chemical injury is associated or caused by alcohol, drugs or neurotoxins.

11. The method or use of claim 9, wherein the acquired brain injury results from compression or blunt trauma to the brain leading to traumatic brain injury (TBI), ischemic injury resulting from a transient ischemic attack (TIA), ischemic stroke or haemorrhagic stroke.

12. The method or use of claim 9, wherein the neurodegenerative condition is characterised by the presence of abnormal protein deposits in the brain, including amyloidopathies, synucleinopathies or taupathies.

13. The method or use of claim 12, wherein the neurodegenerative condition or disorder is selected from the group consisting of: Alzheimer's disease (AD), Lewy-bodies disease (Dementia with Lewy bodies (DLB)), Huntington's disease, Creutzfeldt-Jakob disease (CJD), Gaucher Disease Type 3, and Parkinson's disease.

14. The method or use of claim 9, wherein the neurodegenerative condition or disorder is selected vascular dementia, frontotemporal dementia or other form of dementia not typically associated with deposition of abnormal protein deposits.

15. The method or use of any one of claims 1 to 6, wherein the aggression, agitation, irritability and/or anger is associated with or caused by one or more of AD, vascular dementia, frontotemporal dementia, mixed dementia or Korsakoff syndrome.

16. The method or use of any one of claims 1 to 6, wherein the aggression, agitation, irritability and/or anger is associated with or caused by AD.

17. The method or use of claim 8, wherein the neurodevelopmental disorder is selected from the group consisting of: Fragile-X Syndrome, X-Linked Intellectual Disability-Hypotonia-Facial Dysmorphism-Aggressive Behaviour Syndrome, Kleefstra Syndrome, Hunters Syndrome (MPS II), ADNP (activity dependent neuroprotector homeobox) syndrome, Rett syndrome, Autism Spectrum Disorder, Prader-Willi syndrome, Angelman Syndrome, Brunner syndrome, Cri du Chat syndrome, Cornelia de Lange syndrome, Smith-Lemli-Opitz syndrome, Smith-Magenis syndrome, Tuberous Sclerosis Complex, CHARGE syndrome, sotos syndrome, attention-deficit/hyperactivity disorder (ADHD), PTEN associated disorder, cerebral palsy, and fetal alcohol spectrum disorder.

18. The method or use of any one of claims 1 to 17, wherein the therapeutically effective amount of the compound of Formula (I), or a pharmaceutically acceptable salt or prodrug thereof, does not cause sedation of the subject receiving treatment.

19. The method or use of any one of claims 1 to 18, wherein the compound of Formula (I), or a pharmaceutically acceptable salt or prodrug thereof, is administered orally, intranasally, systemically (e.g., subcutaneously, intramuscularly, intraperitoneally, intravenously) or rectally or is for oral, intranasal, systemic or rectal administration.

20. A kit comprising an effective amount of a compound of Formula (I) or a pharmaceutically acceptable salt and/or prodrug thereof, wherein the kit includes written instructions for performing the methods of any one of claims 1 to 19.
